(19) United States
(12) Patent Application Publication
(10) Pub. No.: US 2007/0099857 A1
(43) Pub. Date: May 3, 2007

(54) Methods and means for inhibiting fever

(76) Inventors: Anders Blomqvist, Linkoping (SE); Per-Johan Jakobsson, Kungsangen (SE); David Engblom, Linkoping (SE)

Correspondence Address: NIXON & VANDERHYE, PC, 901 NORTH GLEBE ROAD, 11TH FLOOR, ARLINGTON, VA, 22203, US

(21) Appl. No.: 10/570,501

(22) PCT Filed: Sep. 1, 2004
(86) PCT No.: PCT/EP04/09734

§ 371 Date: Dec. 5, 2006

Related U.S. Application Data
(60) Provisional application No. 60/501,262, filed on Sep. 9, 2003.

(51) Int. Cl. A61K 48/00; A61K 31/739; C12Q 1/68

(52) U.S. Cl. 514/44; 435/6; 514/54; 514/165

(57) ABSTRACT

Microsomal prostaglandin E synthase-1 (m PGES-1) catalyses the terminal step in the synthesis of prostaglandin E₂ (PGE₂) in the brain. In the absence of mPGES-1, animals do not develop immune-induced fever, but retain a normal pyretic response to central administration of PGE₂. Inhibitors of PGES-1 can be used for treating fever and other PGE₂-dependent reactions. Assays for inhibitors of mPGES-1 are provided.

The present invention relates to treating fever by inhibiting synthesis of prostaglandin E₂ (PGE₂). PGE₂ is synthesised in response to inflammatory cytokines and mediates inflammatory responses including fever and pain. Inhibitors of PGE₂ production may be used in treatment of inflammation, including fever and other PGE₂-dependent reactions. Assay methods for identifying such inhibitors are provided.

Fever is close to an obligatory sign of inflammatory and infectious processes (Elmquist, J. K., Scammell, T. E. & Saper, C. B. Trends. Neurosci. 20, 565-570 (1997)). Several different mechanisms by which peripherally released inflammatory messengers influence the brain to produce fever have been suggested, including the direct action of cytokines on thermoregulatory neurons in the brain as well as indirect effects via peripheral nerves or neurons of the circumventricular organs (Elmquist, J. K., Scammell, T. E. & Saper, C. B. Trends. Neurosci. 20, 565-570 (1997); Engblom, D. et al. J. Mol. Med. 80, 5-15 (2002)). However, accumulating evidence indicates that fever is produced by centrally released PGE₂ that acts on EP receptor expressing neurons in the median preoptic region of the hypothalamus (Elmquist, J. K., Scammell, T. E. & Saper, C. B. Trends. Neurosci. 20, 565-570 (1997); Dinarello, C. A., Gatti, S. & Bartfai, T. Curr. Biol. 9, R147-150 (1999)). The biosynthesis of PGE₂ involves enzymatic action of cyclooxygenase cox-1 or cox-2, which converts arachidonic acid (AA) into prostaglandin endoperoxide H₂ (PGH₂). Subsequently, PGH₂ can be metabolized into PGE₂.

Every day millions of people use anti-pyretic medications, known to ubiquitously inhibit the formation of cyclooxygenase (cox)-derived prostaglandins, to alleviate fever. The role of prostaglandins in inflammation and inflammatory diseases such as arthritis has been documented through the use of various cox-inhibitors, particularly nonsteroidal anti-inflammatory drugs (NSAIDs) including aspirin (Vane & Botting (1998) American J. of Med. 104(3A), 2S-8S). Anti-pyretic medication is the most sold category of drugs. Unfortunately, use of such drugs can have side effects, such as ventricular ulcers, kidney problems and cardiovascular problems. At least some of the drugs side effects may arise because of their interference with synthesis of other cox-dependent prostanoids, such as prostacyclin.

Several reports have demonstrated significant anti-tumour effects by NSAIDs on colorectal cancer (Giovannucci et al. Anals of Internal Medicine 121, 241-6 (1994); Giardiello et al. European Journal of Cancer 31A, 1071 (1995); Williams et al. Journal of Clinical Investigation 100, 1325-9 (1997)). PGE promotes cancer cell proliferation (Qiao et al. Biochimica et Biophysica Acta 1258, 215-23 (1995)) as well as inhibiting programmed cell death (Ottonello et al. Experimental Hematology 26, 895-902 (1998); Goetzl et al. Journal of Immunology 154, 1041-7 (1995)), overall resulting in support of cancer cell growth (Sheng et al. Cancer Research 58, 362-6 (1998)). Inhibition of PGE formation thus leads to slower proliferation in combination with increased apoptosis of the cancer cell population. This inhibiting effect of NSAIDs has also been observed in other cancer conditions such as non-small cell lung cancer (Hida et al. Anticancer Research 18, 775-82 (1998)).

Prostaglandins have also been implicated in Alzheimer's disease. Several clinical trials have demonstrated that users of NSAIDs experience as little as one half of the risk of acquiring Alzheimer's disease (Dubois et al. Faseb J. 12, 1063-1073 (1998)). Consistent with this, other observations suggest that inflammatory processes may contribute to this disease (Aisen Gerontology 43, 143-9 (1997)).

Cox-1 is constitutively expressed in many cells and tissues such as platelets, endothelium, stomach and kidney whereas the cox-2 protein can be induced by proinflammatory cytokines like interleukin-1β (IL-1β) at sites of inflammation. For reviews on cox see Smith, W. Advances in Experimental Medicine & Biology 400B, 989-1011 (1997); Herschman, H. R. Biochimica et Biophysica Acta 1299, 125-40 (1996); Dubois, R., et al. Faseb J. 12, 1063-1073 (1998). Downstream of the cyclooxygenases, their product PGH₂ can be further metabolized into the various physiologically important eicosanoids e.g. PGF_{2α}, PGE₂, PGD₂, PGI₂ (prostacyclin) and thromboxane (TX) A₂ (Smith, W. L. Am. J. Physiol. 263, F181-F191 (1992)).

While conversion of PGH₂ into PGE₂ was long assumed to be non-enzymatic, several PGE₂-synthesizing enzymes have now been identified (Jakobsson et al. Proc. Natl. Acad. Sci. USA 96, 7220-7225 (1999); Tanioka et al. J. Biol. Chem. 275, 32775-32782 (2000); Tanikawa et al. Biochem. Biophys. Res. Commun. 291, 884-889 (2002)), among them microsomal prostaglandin E synthase-1 (mPGES-1) (Jakobsson et al., Proc. Natl. Acad. Sci. USA 96, 7220-7225 (1999)), a membrane associated glutathione-dependent enzyme of the MAPEG family.

Jakobsson, Samuelsson and Morgenstern showed that human PGE synthase is a member of a protein superfamily consisting of membrane associated 14-18 kDa proteins involved in eicosanoid and glutathione metabolism (WO 00/28022). That application includes nucleotide and amino acid sequences of human mPGES-1, where the amino acid sequence SEQ ID NO: 2 is encoded by the human nucleotide sequence SEQ ID NO: 1. References herein to SEQ ID NO: 1 and SEQ ID NO: 2 are to SEQ ID NOS 1 and 2, respectively, as disclosed in WO 00/28022.

The human cDNA sequence of mPGES-1 as well as the predicted amino acid sequence were deposited in 1997 in public databases under the name of MGST1-L1 (GenBank accession number AF027740) as well as a p53 induced PIG12 (GenBank accession number AF010316).

The cDNA sequence (SEQ ID NO: 9) and translated amino acid sequence (SEQ ID NO: 10) of mouse mPGES-1 have been deposited under accession number NM_{—}022415 (Mus musculus prostaglandin E synthase, designated “Ptges”).

mPGES-1 is induced in brain endothelial cells upon immune challenge (Ek, M. et al. Nature 410, 430-431 (2001); Yamagata, K. et al. J. Neurosci. 21, 2669-2677 (2001); Engblom, D. et al. J. Comp. Neurol. 452, 205-214 (2002)) in a temporal pattern that fits with induced increase in intracerebral PGE₂ levels and appearance of fever (Inoue et al. Neurosci. Res. 44, 51-61 (2002)). The concomitant expression in brain endothelial cells of inducible cox-2 and of receptors for proinflammatory cytokines (Ek, M. et al. Nature 410, 430-431 (2001)) suggests that these cells display components enabling a blood-born inflammatory signal to stimulate the production of PGE₂, which subsequently may diffuse into the brain because of its small size and lipophilic properties.

mPGES-1 is also consitutively expressed in brain parenchyma. However, its expression is very sparse, being restricted to motor neuron groups (Engblom, D. et al. J. Comp. Neurol. 452, 205-214 (2002)).

An inducible PGE synthase activity has also been described in lipopolysaccharide (LPS)-stimulated rat peritoneal macrophages, which coincides with cox-2 expression and changes the product formation in favour of PGE₂ (Naraba, H. et al. Journal of Immunology 160, 2974-82 (1998); Matsumoto, H., et al. Biochemical & Biophysical Research Communications 230, 110-4 (1997)).

mPGES-1 is expressed ubiquitously in brain venules upon immune challenge (Ek, M. et al. Nature 410, 430-431 (2001); Yamagata, K. et al. J. Neurosci. 21, 2669-2677 (2001)). Dual induction of Cox-2 and mPGES in IL-1 receptor expressing endothelial cells in brain venules and capillaries has also been shown in an adjuvant-induced arthritis model (Engblom, D. et al. J. Comp. Neurol. 452, 205-214 (2002)). The specificity of cellular and functional responses elicited by the PGE₂ is dependent and mediated by specific subgroups of PGE₂ receptors (Engblom, D. et al. J. Mol. Med. 80, 5-15 (2002)). This is shown by unresponsiveness to the pyrogenic action of PGE₂ by EP₃ receptor^{-/-} mice (Ushikubi, F. et al. Nature 395, 281-284 (1998)).

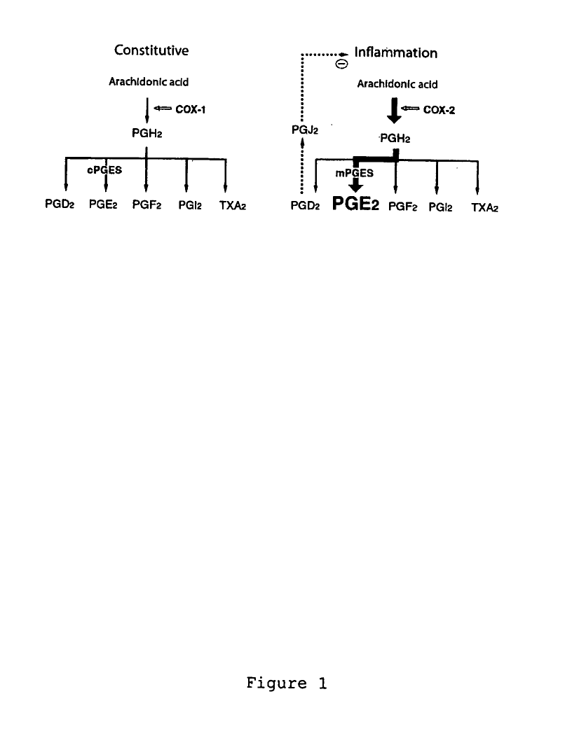

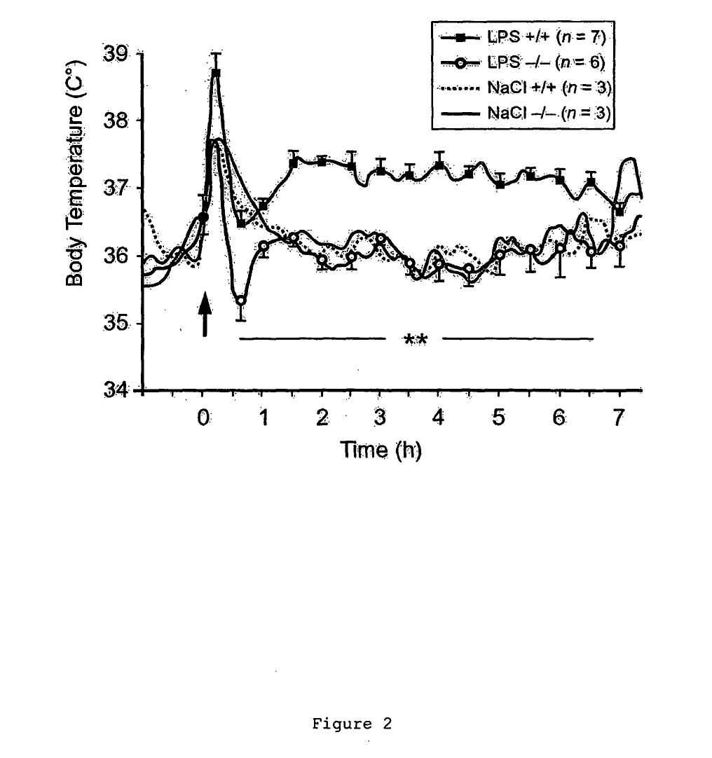

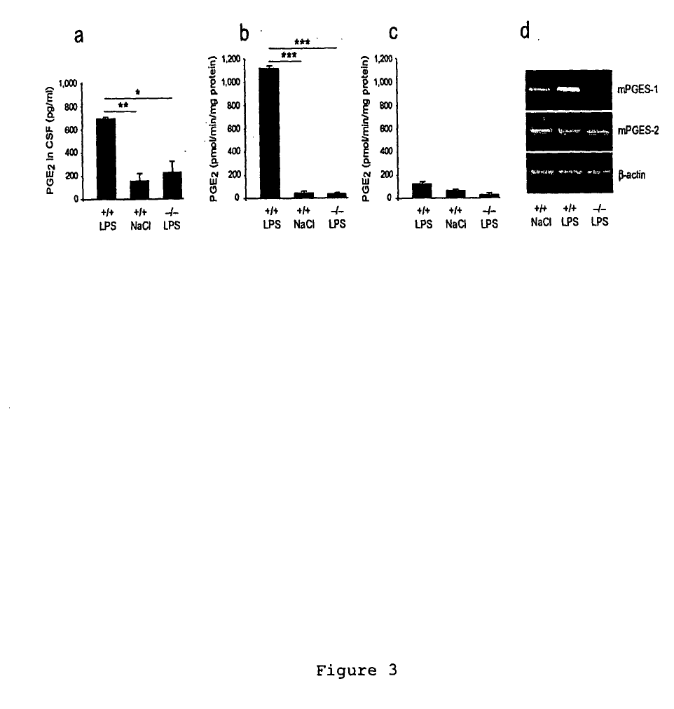

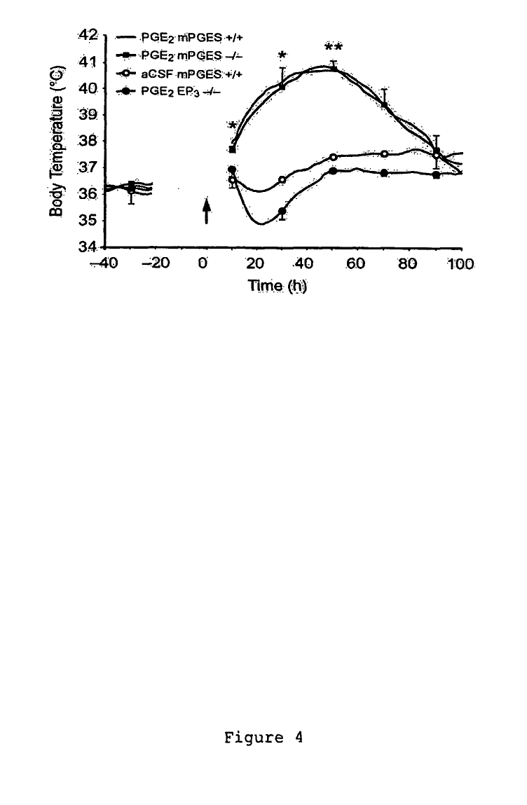

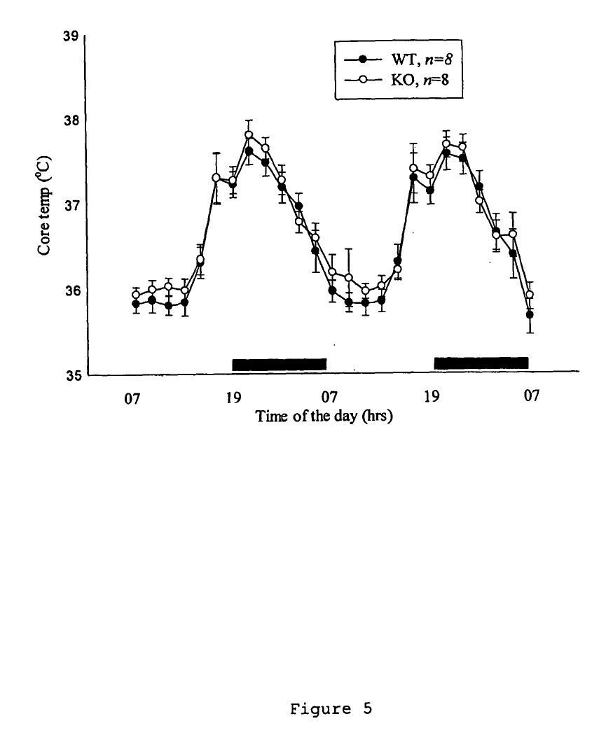

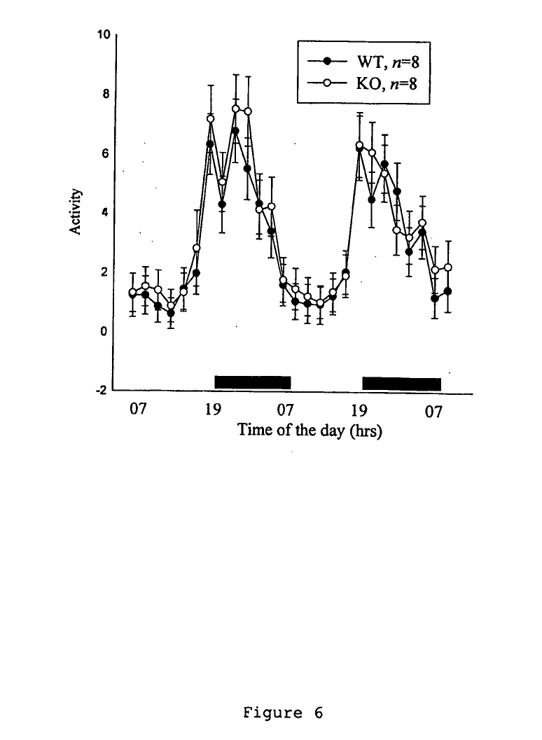

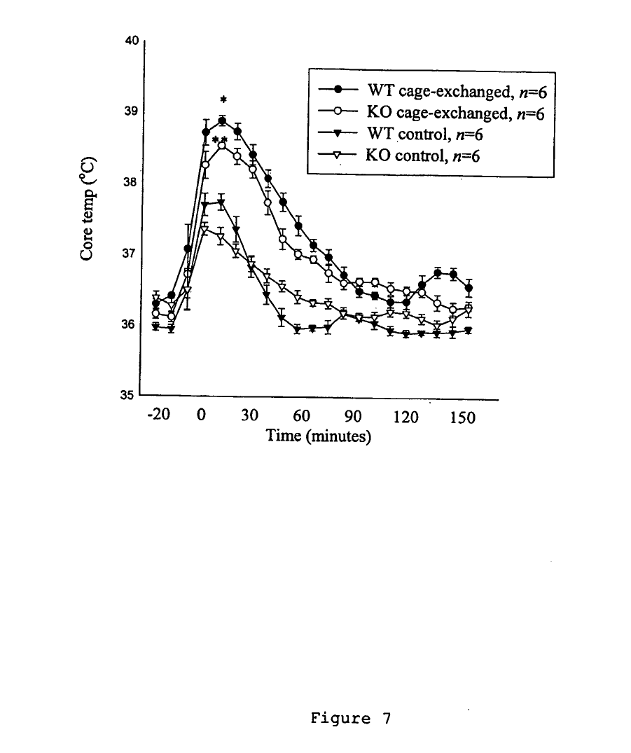

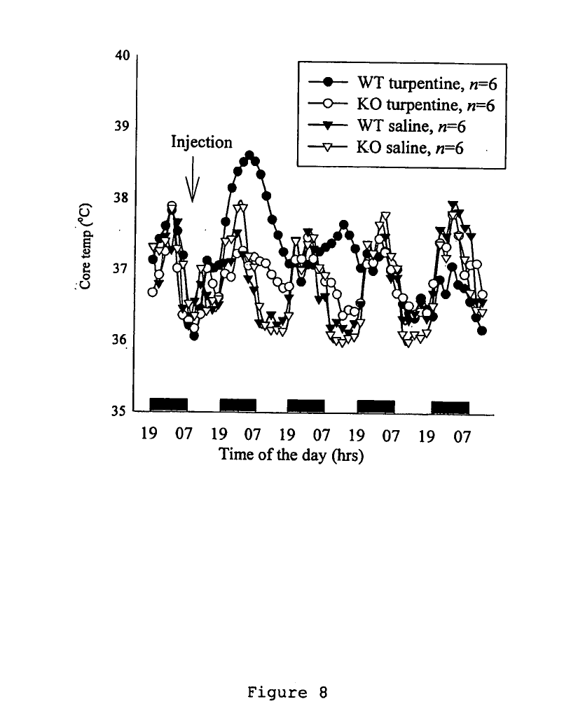

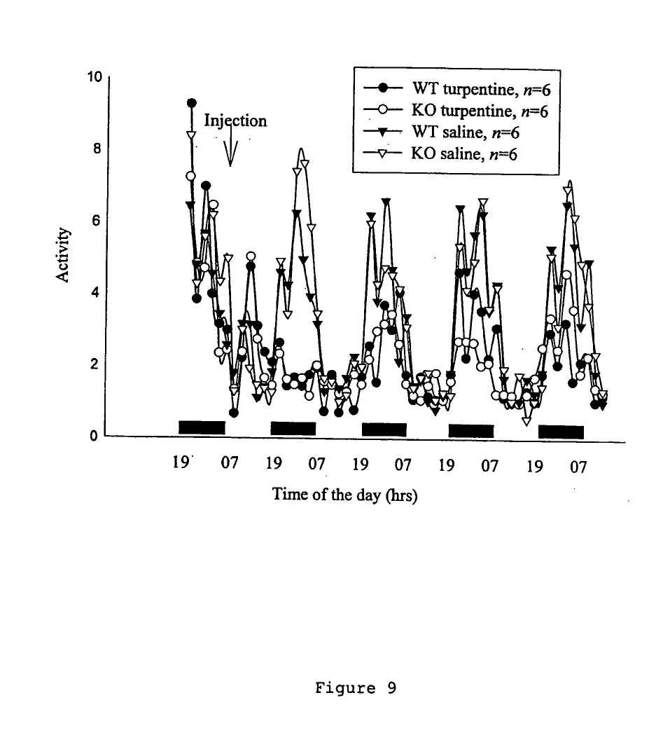

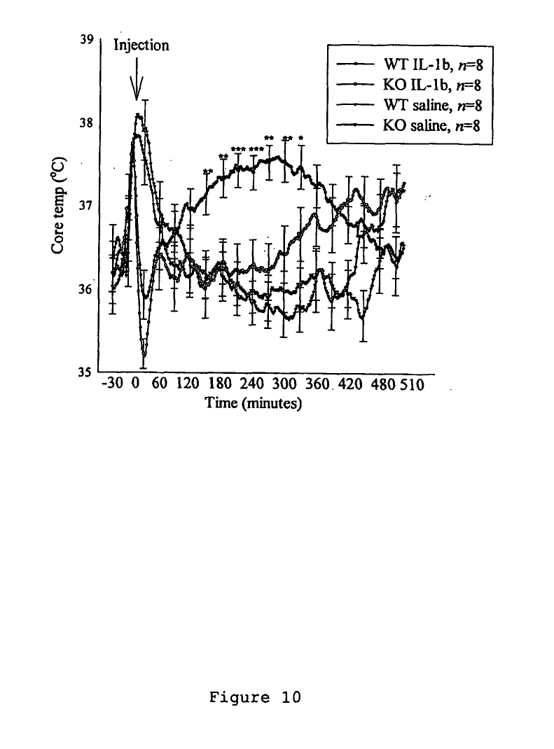

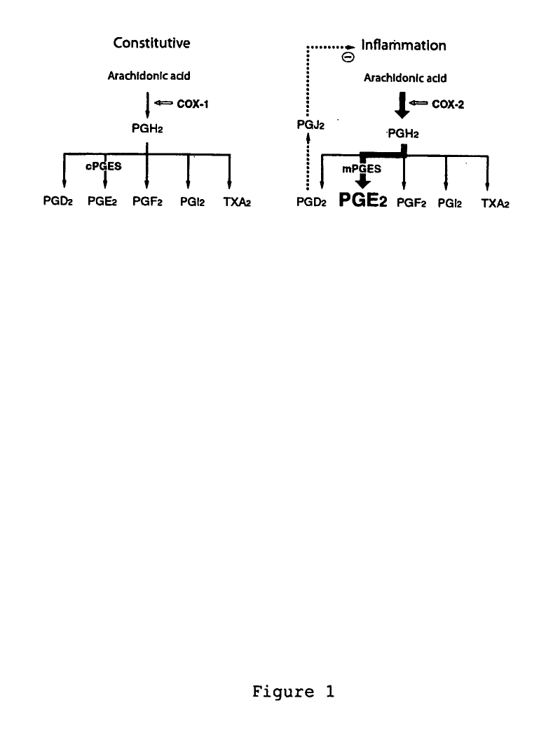

Constitutive production of PGE₂ has been shown to be subserved preferentially by the enzymes cytosolic PGE synthase (cPGES, FIG. 1) or mPGES-2 (Tanikawa et al. Biochem. Biophys. Res. Commun. 291, 884-889 (2002)), which both are located downstream of cox-1 and cox-2, and which are not upregulated in the brain (as shown herein) or in peripheral tissues (Murakami et al. J. Biol. Chem. June 30 [Epub ahead of print] (2003); Claveau et al. J. Immunol. 170, 4738-4744 (2003)). However, a threefold upregulation of cPGES in brain was reported after intravenous injection of a high dose of LPS (Tanioka et al. J. Biol. Chem. 275, 32775-32782 (2000)). cPGES is understood to be involved in “house-keeping” functions, and to preferentially couple to Cox-1.

We show herein that mice deficient in mPGES-1 show no fever and no central PGE₂ synthesis after immune stimulation, but the mice display intact pyretic capacity in response to centrally administered PGE₂. Based on the findings disclosed herein, we conclude that mPGES-1 is the central switch during immune-induced pyresis. Our data indicate that other PGE-synthases are not involved in PGE₂ production following inflammatory stimulation, and we propose that fever could be treated, reduced or prevented through inhibition of mPGES-1. We identify mPGES-1 as a novel and specific drug target for the treatment of fever and other acute phase PGE₂-dependent reactions elicited by the brain.

The present invention relates to inhibition of mPGES-1 to alleviate, treat, reduce or prevent fever (pyretic response) and other centrally elicited acute phase reactions, and also to methods of identifying inhibitors of mPGES-1.

Our results indicate that inhibition of mPGES-1. may be used as anti-pyretic treatment, like the presently available cox inhibitors. However, because mPGES-1 is the terminal isomerase in the PGE₂ synthetic pathway, its inhibition should have more selective effects compared to inhibition of enzymes earlier in the pathway and should interfere almost exclusively with inflammation-induced PGE₂, leaving not only the constitutive PGE₂ synthesis unaffected, but also the synthesis of other cox-derived prostanoids (FIG. 1), some of which may have anti-inflammatory properties (Gilroy et al. Nat. Med. 5 698-701 1999).

As noted, constitutive expression of mPGES-1 in the brain parenchyma is very sparse. This is in stark contrast to the widespread expression among large numbers of cell groups of cox-2 (Breder et al. J. Comp. Neurol. 355, 296-315 (1995)), which is the target of the new generation anti-inflammatory NSAIDs. Furthermore, constitutive PGE₂ synthesis by cPGES-1 and/or mPGES-2 are unaffected by inhibition of mPGES-1.

Therefore, inhibition of mPGES-1accordingly to the present invention not only leaves synthesis of other cox-derived prostanoids unaffected, but also leaves constitutive PGE₂ synthesis substantially unaffected. Effects of inhibiting mPGES-1are therefore highly selective, targeting only induced PGE₂ synthesis such as PGE₂ induced by an inflammatory stimulus.

Thus, the present invention allows for more selective inhibition of PGE₂ synthesis by inhibiting mPGES-1, avoiding many of the side effects associated with presently available drugs. Specific removal of PGE₂ by inhibition of mPGES-1 may be used to provide control of inflammatory reactions with fewer side effects in comparison with presently used NSAIDs.

Because of the importance of PGE₂ in inflammation and other contexts of medical significance, important aspects of the invention are concerned with identifying substances which are able to block, reduce or inhibit PGE₂ production by inhibiting mPGES-1 activity. The invention provides methods of screening for substances that inhibit production of PGE₂ and inhibit, treat (including preventative treatment) or alleviate inflammatory or inflammation-induced reactions, especially fever (pyretic response). Screening methods of the invention may involve determining the ability of candidate inhibitors to reduce PGE₂-induced reactions, especially fever, in an animal.

Methods of the invention may be used to identify inhibitors of mPGES-1 that inhibit pyretic response, especially pyretic response to an inflammatory stimulus.

The present invention relates to PGE₂-dependent inflammatory responses, especially fever. In addition to inflammation-induced fever, other mechanisms can result in hyperthermia, such as acute stress, and external heating. Such hyperthermia is, however, generally not considered to be “fever”, and it is not PGE₂-dependent. Therefore, such hyperthermia is not within the scope of the present invention. The present invention relates to fever induced by inflammation or an inflammatory stimulus. As noted above, PGE₂-mediated responses are associated with certain diseases including cancer and Alzheimer's disease, and the present invention may be used in relation to inflammatory response and/or fever associated with such diseases. Fever in the present invention includes fever associated with cancer, which is elicited by similar peripheral mechanisms as inflammation-induced fever.

PGE₂ receptors are expressed by neurons in several brain regions that are associated with other inflammation-induced reactions, such as anorexia (decreased food intake) and hyperalgesia (increased pain sensitivity), as well as hormonal stress responses. This provides an indication that these reactions are also elicited by PGE₂ produced in the brain. For example, it has been shown that neurons in the brain stem parabrachial nucleus, a major nociceptive and visceroreceptive relay in rodents, express EP-receptors (Engblom et al. Neurosci. Lett. 281, 163-166 (2000)) that peripheral immune challenge activates EP-receptor bearing neurons in this nucleus (Engblom et al., J. Comp. Neurol. 440, 378-386 (2001)), and that the EP-receptor bearing neurons express the peptide messengers (Engblom et al., Soc. Neurosci. Abstr. 28, 867.14 (2002)) implicated in nociceptive transmission through this nucleus (Hermanson, Linköping Univ. Med. Diss. No. 514, 1997; Malick et al., PNAS 98, 9930-9935 (2001)).

Aspects of the invention may therefore also be applied to inhibiting PGE₂-induced reactions other than fever, for example anorexia, hyperalgesia and hormonal stress responses. This should be borne in mind in relation to the aspects of the invention which will now be described in relation to inhibition of fever (pyretic response), as a preferred embodiment.

One aspect of the present invention provides a method of identifying an inhibitor of fever, comprising
administering a candidate inhibitor to a test animal, wherein the candidate inhibitor is a substance that inhibits microsomal PGE synthase-1 activity; and
determining the level of febrile response to a stimulus in the test animal compared to the level of febrile response to the stimulus in a control animal,
whereby a lower febrile response in the test animal than in the control animal indicates that the candidate inhibitor inhibits fever.

No candidate inhibitor is administered to the control animal. Preferably, a control administration is given to the control animal, such as physiological saline solution, and is preferably administered by the same route as administration of the test substance to the test animal.

The test animal is normally a mammal, preferably a rodent such as a mouse. The stimulus in the test animal is normally an inflammatory or immune stimulus designed to generate an inflammatory response. For example, the stimulus may be LPS, but it may also be one or more cytokines, such as interleukin-1 beta, live bacteria or other microorganisms, such as viruses or parasites, or an experimentally induced aseptic peripheral lesion, such as a lesion elicited by an externally administered tissue damaging substance (e.g. turpentine, carrageenan, Freund's adjuvant). The stimulus may also be an experimental model mimicking septic peritonitis (e.g. an iliocecal ligature and puncture) or mimicking prolonged inflammatory disease, such as a model of autoimmune arthritis. Thus, in some embodiments the stimulus may be present in the animal prior to administration of the candidate inhibitor, especially where the stimulus comprises a disease model in the animal. Alternatively or additionally, the stimulus may be administered to the animal or induced in the animal following administration of the candidate inhibitor.

The method may comprise identifying the candidate inhibitor as an inhibitor of fever. This involves determining, observing or detecting a lower febrile response in the test animal compared with the control animal. For example, body temperature of the animals may be monitored, wherein a rise in body temperature may indicate fever. Accordingly, no rise, a lower rise or a less-sustained rise in body temperature of the test animal compared with the control animal may indicate a lower febrile response in the test animal. For further details of techniques applicable to the invention generally, see the experimental part below.

The candidate inhibitor administered to the test animal is a substance previously identified as, or believed to be, an inhibitor of mPGES-1. Known inhibitors of mPGES-1 may be used in methods of the invention, as may compounds developed from known inhibitors. Candidate inhibitors may be identified using any suitable assay method, examples of which are described herein.

Preferably, the candidate inhibitor does not significantly inhibit another enzyme such as a cyclooxygenase or another PGE synthase. Preferably, the candidate inhibitor is a specific inhibitor of mPGES-1, meaning that it inhibits mPGES-1 while not significantly inhibiting other enzymes especially cyclooxygenases and other PGE synthases.

To date there are no known specific inhibitors of mPGES-1. However a number of compounds have been found to inhibit the enzyme, including leukotriene C4, NS-398, sulindac sulfide with IC50 values of 5, 20 and 80 μM, respectively (Thoren et al. Eur. J. Biochem. 267, 6428 (2000)). Also, 15-deoxy-Δ12, 14-PGJ₂, arachidonic acid, docosahexaenoic acid, eicosapentaenoic acid and MK886 were all reported to inhibit mPGES with similar IC50 values of 0.3 μM (Mancini et al. J. Biol. Chem., 276, 4469 (2001)), (Quraishi et al. Biochem. Pharmacol., 63, 1183 (2002)). The lack of specificity of these compounds makes them unsuitable for in vivo testing according to the present invention. The invention provides methods of developing candidate inhibitors, e.g. developing mimetics, and of screening for inhibitors of mPGES-1, that may be used to develop specific inhibitors of mPGES-1.

Methods of the invention may comprise identifying a substance that inhibits mPGES-1 (a candidate inhibitor), preferably a substance that inhibits mPGES-1specifically or at least does not inhibit a cox or another PGE synthase, and then administering the substance to a test animal as described herein. The animal testing stage may be used to confirm whether the candidate inhibitor inhibits fever, to determine the extent of inhibition of fever, and/or to determine whether the candidate inhibitor gives rise to side effects.

Preferably, the assay method includes a method of screening for a candidate inhibitor of mPGES-1 that specifically inhibits of mPGES-1 activity. The assay method may comprise determining whether a candidate inhibitor or test substance inhibits another enzyme such as a cox or a PGE synthase other than mPGES-1. The method may comprise:
incubating a test substance, or a candidate inhibitor previously found to inhibit mPGES-1 activity, in the presence of an enzyme other than mPGES-1, preferably a cyclooxygenase (e.g. cox-1 or cox-2) or a PGE synthase other than mPGES-1 (e.g. mPGES-2 or cPGES) under conditions in which the enzymes normally catalyse a reaction producing a product; and
determining production of the product.

The method normally comprises incubating the test substance or candidate inhibitor with the enzyme and a substrate for the enzyme. The substrate may be a physiological substrate such as AA for cox, or it may be a modified or non-physiological substrate, such as a substrate designed to give rise to a detectable (e.g. coloured) product in the enzymatic reaction.

Determination of reduced production of product compared with a control experiment in which the test substance or candidate inhibitor is not applied indicates that the test substance or candidate inhibitor inhibits the enzyme used and is not a specific inhibitor of mPGES-1. Thus, production of the product in the presence of the test substance may be compared with production of the product in the absence of the test substance. A lower level of product, or a lower rate of product formation indicates that the test substance or candidate inhibitor inhibits the enzyme activity. If production of the product is not reduced, then the test substance or candidate inhibitor is not an inhibitor of the enzyme and may be a specific inhibitor of mPGES-1. The method of the invention may comprise determining that production of the product is not reduced compared with production in the absence of the test substance or candidate inhibitor.

Assay methods of the invention may comprise one or more such methods to determine whether a test substance or candidate inhibitor inhibits enzymes other than mPGES-1. The test substance or candidate inhibitor may be used in an array of such assays to determine whether or not it inhibits a number of different enzymes including mPGES-1 and other enzymes such as cycloxygenases and other PGE synthases. Inhibition of mPGES-1 and no inhibition of other enzymes indicates that the test substance or candidate inhibitor is a specific inhibitor of mPGES-1 and is therefore a highly preferred candidate inhibitor for animal testing according to the present invention.

The assay method may include an initial screen for substances that might inhibit mPGES-1. For example, an assay method for identifying a candidate inhibitor may comprise:
(a) bringing into contact an mPGES-1 polypeptide and a putative binding molecule or other test substance; and
(b) determining interaction or binding between the polypeptide and the test substance.

Determination of the ability of a test substance to interact and/or bind with mPGES-1 may be used to identify that test substance as a possible inhibitor of mPGES-1 activity. The method may comprise detecting or observing interaction or binding, and then using that test substance in a further assay method to determine whether it inhibits mPGES-1 activity.

The precise format of assays of the invention may be varied by those of skill in the art using routine skill and knowledge. For example, interaction between polypeptides or peptides may be studied in vitro by labelling one with a detectable label and bringing it into contact with the other which has been immobilised on a solid support. Suitable detectable labels include ³⁵S-methionine which may be incorporated into recombinantly produced peptides and polypeptides. Recombinantly produced peptides and polypeptides may also be expressed as a fusion protein containing an epitope which can be labelled with an antibody.

The protein or peptide that is immobilized on a solid support may be immobilized using an antibody against that protein bound to a solid support or via other technologies which are known per se. A preferred in vitro interaction may utilise a fusion protein including glutathione-S-transferase (GST). This may be immobilized on glutathione agarose beads. In an in vitro assay format of the type described above a test compound can be assayed by determining its ability to diminish the amount of labelled peptide or polypeptide which binds to the immobilized GST-fusion polypeptide. This may be determined by fractionating the glutathione-agarose beads by SDS-polyacrylamide gel electrophoresis. Alternatively, the beads may be rinsed to remove unbound protein and the amount of protein which has bound can be determined by counting the amount of label present in, for example, a suitable scintillation counter.

Generally, the identification of ability of a test substance to bind or interact with mPGES-1 and its identification as a candidate inhibitor is followed by one or more further assay steps involving determination of whether or not the test substance is able to inhibit mPGES-1 activity. Naturally, assays involving determination of ability of a test substance to inhibit mPGES-1 may be performed where there is no knowledge about whether the test substance can bind or interact with mPGES-1, but a prior binding/interaction assay may be used as a screen to test a large number of substances, reducing the number of potential. inhibitors to a more manageable level for a functional assay involving determination of ability to inhibit mPGES-1 activity.

A further possibility for an assay for inhibitors is testing ability of a substance to affect PGE₂ production by a suitable cell line expressing mPGES-1 (either naturally or recombinantly). An assay according to the present invention may also take the form of an in vivo assay. The in vivo assay may be performed in a cell line such as a yeast strain in which the relevant polypeptides or peptides are expressed from one or more vectors introduced into the cell.

A still further possibility for an assay is testing ability of a substance to affect PGE₂ production by an impure protein preparation including mPGES-1 (whether human or other mammalian). A preferred assay of the invention includes determining the ability of a test substance to inhibit mPGES-1 activity of an isolated/purified mPGES-1 polypeptide (including a full-length mPGES-1 or an active portion thereof).

A method of screening for a substance which inhibits activity of an mPGES-1 polypeptide (i.e. an inhibitor of mPGES-1) may include contacting one or more test substances with the polypeptide in a suitable reaction medium, testing the activity of the treated polypeptide and comparing that activity with the activity of the polypeptide in comparable reaction medium untreated with the test substance or substances. A difference in activity between the treated and untreated polypeptides is indicative of a modulating effect of the relevant test substance or substances.

The assay method may comprise:
(a) incubating an mPGES-1 polypeptide and a test compound in the presence of reduced glutathione and PGH₂ under conditions in which PGE₂ is normally produced; and
(b) determining production of PGE₂.

PGH₂ substrate for mPGES-1 may be provided by incubation of COX-2 and AA, so these may be provided in the assay medium in order to provide PGH₂.

Furthermore, mPGES-1 catalyses sterospecific formation of 9-keto, 11α hydroxy prostaglandin from the cyclic endoperoxide and so other substrates of mPGES-1 may be used in determination of mPGES-1 activity, and the effect on that activity of a test compound, by determination of production of the appropriate product.

| Substrate | Product |
|---|---|
| PGH₂ | PGE₂ |
| PGH₁ | PGE₁ |
| PGH₃ | PGE₃ |
| PGG₂ | 15(S)hydroperoxy PGE₂ |
| PGG₁ | 15(S)hydroperoxy PGE₁ |
| PGG₃ | 15(S)hydroperoxy PGE₃ |

Thus, the assay method may comprise:
(a) incubating an mPGES-1 polypeptide and a test compound in the presence of a cyclic endoperoxide substrate of mPGES-1 under conditions in which mPGES-1 normally catalyses conversion of the cyclic endoperoxide substrate into a product which is the 9-keto, 11α hydroxy form of the substrate; and
(b) determining production of said product.

As noted, the substrate may be any of those discussed above, or any other suitable substrate at the disposal of the skilled person. It may be PGH₂, with the product then being PGE₂.

In assay methods of the invention, production of product can be measured by quantifying level of substrate and/or by quantifying level of product. Any remaining substrate at the end of the assay or the time of terminating the assay reaction, can be converted into 12-hydroxyheptadeca trienoic acid and malon dialdehyde or PGF-2α by adding iron chloride or stannous chloride, respectively. Thus, the amounts of these compounds then reflect indirectly the formation of PGE₂. Quantifying these compounds is a means of determining production of the product, by quantifying the amount of remaining substrate. The greater the level of remaining substrate, the lower the level of production of the product.

An inhibitor of mPGES-1 may be identified (or a candidate substance suspected of being a PGE synthase inhibitor may be confirmed as such) by determination of reduced production of PGE₂ or other product (depending on the substrate used) compared with a control experiment in which the test substance is not applied. Thus, production of the product in the presence of the test substance may be compared with production of the product in the absence of the test substance. A lower level of product, or a lower rate of product formation indicates that the test substance inhibits mPGES-1. activity. Thus, the test substance may be identified as a candidate inhibitor of fever.

Product determination may employ HPLC, UV spectrometry, radioactivity detection, or RIA (such as a commercially available RIA kit for detection of PGE). Product formation may be analysed by gas chromatography (GC) or mass spectrometry (MS), or TLC with radioactivity scanning.

An inhibitor of mPGES-1 may inhibit mPGES-1 by inhibiting its expression from encoding DNA.

Accordingly, assay methods of the invention may comprise identifying a candidate inhibitor of fever, wherein the method comprises screening for a substance able to reduce or inhibit expression of a gene encoding mPGES-1, comprising:
(a) contacting DNA containing the promoter of said gene with a test substance, wherein the promoter is operably linked to a gene
(b) determining the level of gene expression from the promoter, and
(c) comparing said level of gene expression in the presence of the test substance with the level of gene expression in the absence of the test substance in comparable conditions,
   wherein a reduced level of gene expression in the presence of the test substance indicates that the test substance is able to inhibit expression of the mPGES-1 gene.

The method may further comprise identifying the test substance as an inhibitor of expression of the mPGES-1 gene, i.e. as a candidate inhibitor of fever according to the present invention.

Thus, step (c) may comprise detecting a reduced level of gene expression in the presence of the test substance compared with the level of gene expression in the absence of the test substance in comparable conditions,
whereby the test substance is identified as a candidate inhibitor of fever.

A reduction in expression of the gene compared with expression of another gene linked to a different promoter indicates specificity of the substance for inhibition of the mPGES-1 promoter.

The method may comprise contacting an expression system, such as a host cell containing the gene promoter operably linked to a gene with the test substance, and determining expression of the gene. The gene may be the mPGES-1 gene itself or it may be a heterologous gene, e.g. a reporter gene. A “reporter gene” is a gene whose encoded product may be assayed following expression, i.e. a gene which “reports” on promoter activity.

By “promoter” is meant a sequence of nucleotides from which transcription may be initiated of DNA operably linked downstream (i.e. in the 3′ direction on the sense strand of double-stranded DNA). The promoter of the mPGES-1 gene may comprise one or more fragments of the sequence under an accession number provided herein, sufficient to promote gene expression. The promoter of a gene may comprise or consist essentially of a sequence of nucleotides 5′ to the gene in the human chromosome, or an equivalent sequence in another species, such as a rat or mouse.

The level of promoter activity is quantifiable for instance by assessment of the amount of mRNA produced by transcription from the promoter or by assessment of the amount of protein product produced by translation of mRNA produced by transcription from the promoter. The amount of a specific mRNA present in an expression system may be determined for example using specific oligonucleotides which are able to hybridise with the mRNA and which are labelled or may be used in a specific amplification reaction such as the polymerase chain reaction (PCR).

PCR comprises steps of denaturation of template nucleic acid (if double-stranded), annealing of primer to target, and polymerisation. The nucleic acid probed or used as template in the amplification reaction may be genomic DNA, cDNA or RNA. Other specific nucleic acid amplification techniques include strand displacement activation, the QB replicase system, the repair chain reaction, the ligase chain reaction and ligation activated transcription. For convenience, and because it is generally preferred, the term PCR is used herein in contexts where other nucleic acid amplification techniques may be applied by those skilled in the art. Unless the context requires otherwise, reference to PCR should be taken to cover use of any suitable nucleic amplification reaction available in the art.

Use of a reporter gene facilitates determination of promoter activity by reference to protein production. The reporter gene preferably encodes an enzyme which catalyses a reaction that produces a detectable signal, preferably a visually detectable signal, such as a coloured product. Many examples are known, including β-galactosidase and luciferase. β-galactosidase activity may be assayed by production of blue colour on substrate, the assay being by eye or by use of a spectrophotometer to measure absorbance. Fluorescence, for example that produced as a result of luciferase activity, may be quantified using a spectrophotometer. Radioactive assays may be used, for instance using chloramphenicol acetyltransferase, which may also be used in non-radioactive assays. The presence and/or amount of gene product resulting from expression from the reporter gene may be determined using a molecule able to bind the product, such as an antibody or fragment thereof. The binding molecule may be labelled directly or indirectly using any standard technique.

A promoter construct may be introduced into a cell line using any suitable technique to produce a stable cell line containing the reporter construct integrated into the genome. The cells may be grown and incubated with test compounds for varying times. The cells may be grown in 96 well plates to facilitate the analysis of large numbers of compounds. The cells may then be washed and the reporter gene expression analysed. For some reporters, such as luciferase the cells will be lysed then analysed.

Those skilled in the art are aware of a multitude of possible reporter genes and assay techniques which may be used to determine gene activity. For more examples, see Sambrook and Russell, Molecular Cloning: a Laboratory Manual: 3rd edition, 2001, Cold Spring Harbor Laboratory Press.

mPGES-1 may be inhibited using antisense or RNA interference (RNAi). A candidate inhibitor or test substance may be an anti-sense oligonucleotide or double stranded RNA corresponding to the mPGES-1 gene or a fragment thereof. Anti-sense oligonucleotides are complementary nucleic acid sequences designed to hybridise to nucleic acid of interest, e.g. pre-mRNA or mature mRNA, thus interfering with the production of polypeptide encoded by a given DNA sequence (e.g. either native polypeptide or a mutant form thereof), so that its expression is reduced or prevented altogether. Anti-sense techniques may be used to target a coding sequence, a control sequence of a gene, e.g. in the 5′ flanking sequence, whereby the antisense oligonucleotides can interfere with control sequences. Anti-sense oligonucleotides may be DNA or RNA and may be of around 14-23 nucleotides, particularly around 15-18 nucleotides, in length. The construction of antisense sequences and their use is described in Peyman and Ulman, 1990; and Crooke, 1992.

Anti-sense nucleic acid molecules can be introduced and targeted to cells using techniques described herein. Other approaches to specific down-regulation of genes are well known, including the use of ribozymes designed to cleave specific nucleic acid sequences. Ribozymes are nuceic acid molecules, actually RNA, which specifically cleave single-stranded RNA, such as mRNA, at defined sequences, and their specificity can be engineered. Hammerhead ribozymes may be preferred because they recognise base sequences of about 11-18 bases in length, and so have greater specificity than ribozymes of the Tetrahymena type which recognise sequences of about 4 bases in length, though the latter type of ribozymes are useful in certain circumstances. References on the use of ribozymes include Marschall, et al. 1994; Hasselhoff, 1988 and Cech, 1988.

An alternative to anti-sense is to use a copy of all or part of the target gene inserted in sense, that is the same, orientation as the target gene, to achieve reduction in expression of the target gene by co-suppression; Angell & Baulcombe 1997; and Voinnet & Baulcombe 1997. Double stranded RNA (dsRNA) has been found to be even more effective in gene silencing than both sense or antisense strands alone (Fire et al. 1998). dsRNA mediated silencing is gene specific and is often termed RNA interference (RNAi).

RNA interference is a two step process. First, dsRNA is cleaved within the cell to yield short interfering RNAs (siRNAs) of about 21-23 nt length with 5′ terminal phosphate and 3′ short overhangs (˜2 nt) The siRNAs target the corresponding mRNA sequence specifically for destruction (Zamore 2001).

RNAi may be also be efficiently induced using chemically synthesized siRNA duplexes of the same structure with 3′-overhang ends (Zamore et al. 2000). Synthetic siRNA duplexes have been shown to specifically suppress expression of endogenous and heterologeous genes in a wide range of mammalian cell lines (Elbashir et al. 2001). See also Fire 1999; Sharp 2001; Hammond et al. 2001; and Tuschl 2001.

Methods of screening for antisense, ribozyme and/or iRNA inhibitors of mPGES-1 expression are further provided by the present invention.

In methods of the invention employing mPGES-1 protein, the entire (full-length) mPGES-1 protein sequence need not be used. Assays of the invention which test for binding between two molecules or test for PGE synthase activity may use fragments or variants. Fragments may be generated and used in any suitable way known to those of skill in the art. Suitable ways of generating fragments include, but are not limited to, recombinant expression of a fragment from encoding DNA. Such fragments may be generated by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Small fragments (e.g. up to about 20 or 30 amino acids) may also be generated using peptide synthesis methods which are well known in the art. Active portions of mPGES-1 may be used in assay methods.

mPGES-1, or an mPGES-1 polypeptide, used according to the invention is preferably human mPGES-1, but it may be from another animal such as another mammal, e.g. a mouse or other rodent. Preferably, the mPGES-1 polypeptide comprises or consists of the amino acid sequence of SEQ ID NO. 2. Preferably, an mPGES-1 polypeptide is an isolated polypeptide.

Isolated polypeptides of the invention will be those as defined herein in isolated form, free or substantially free of material with which it is naturally associated such as other polypeptides with which it is found in the cell. The polypeptides may be formulated with diluents or adjuvant and still for practical purposes be isolated—for example the polypeptides will normally be mixed with gelatine or other carriers if used to coat microtitre plates for use in immunoassays. The polypeptides may be glycosylated, either naturally or by systems of heterologous eukaryotic cells, or they may be (for example if produced by expression in a prokaryotic cell) unglycosylated. The term “lacking native glycosylation” may be used with reference to a polypeptide which either has no glycosylation (e.g. following production in a prokaryotic cell) or has a pattern of glycosylation that is not the native pattern, e.g. as conferred by expression in a particular host cell type (which may be CHO cells).

An mPGES-1 polypeptide may be modified for example by the addition of a signal sequence to promote their secretion from a cell or of histidine residues to assist their purification. Fusion proteins may be generated that incorporate (e.g.) six histidine residues at either the N-terminus or C-terminus of the recombinant protein. Such a histidine tag may be used for purification of the protein by using commercially available columns which contain a metal ion, either nickel or cobalt (Clontech, Palo Alto, Calif., USA). These tags also serve for detecting the protein using commercially available monoclonal antibodies directed against the six histidine residues (Clontech, Palo Alto, Calif., USA).

Polypeptides which are amino acid sequence variants, alleles, derivatives or mutants may also be used in methods according to the present invention, such forms having at least 70% sequence identity, for example at least 80%, 90%, 95%, 98% or 99% sequence identity to SEQ ID NO. 2. A polypeptide which is a variant, allele, derivative or mutant may have an amino acid sequence which differs from that given in SEQ ID NO. 2 by one or more of addition, substitution, deletion and insertion of one or more (such as from 1 to 20, for example 2, 3, 4, or 5 to 10) amino acids.

The amino acid sequence of SEQ ID NO. 2 is encoded by the human nucleotide sequence of SEQ ID NO. 1. mPGES-1 polypeptides used in methods of the invention include those encoded by alleles of the human sequence, and homologues of other mammals, particularly primates, as well as fragments of such polypeptides as discussed further below. The primary sequence of the PGE synthase protein will be substantially similar to that of SEQ ID NO. 2 and may be determined by routine techniques available to those of skill in the art. In essence, such techniques include using polynucleotides derived from SEQ ID NO. 1 as probes to recover and to determine the sequence of the PGE synthase gene in other species.

An “active portion” of an mPGES-1 polypeptide may be used in methods of the invention. An active portion means a peptide which is less than the full length polypeptide, but which retains its essential biological activity. In particular, the active portion retains the ability to catalyse PGE₂ synthesis from PGH₂ in the presence of glutathione.

Suitable active portions thus include the central segment of SEQ ID NO. 2, e.g. between about residues 30-130. The relevant catalytic region of the PGE synthase protein is expected to be in the central segment of SEQ ID NO. 2 based on analogy with MGST1 and LTC₄ synthase: amino acids 1-41 can be removed from MGST1 by proteolysis without loss of function (Andersson et al. Biochim. Biophys. Acta 1204, 298-304 (1994)); C-terminal segments can be exchanged between LTC₄ synthase and FLAP without alteration of protein function (Lam et al., J. Biol. Chem. 272, 13923-13928 (1997)).

One active portion of the invention includes or consists of amino acids 30-152 of SEQ ID NO. 2. Another active portion includes or consists of amino acids 1-130 of SEQ ID NO. 2. A still further active portion includes or consists of amino acids 30-130 of SEQ ID NO. 2.

The mPGES-1 polypeptide may include heterologous amino acids, such as an identifiable sequence or domain of another protein, or a histidine tag or other tag sequence. It may consist of an active portion of mPGES-1.

An mPGES-1 polypeptide may be labelled with a revealing label. The revealing label may be any suitable label which allows the polypeptide to be detected. Suitable labels include radioisotopes, e.g. ¹²⁵I, enzymes, antibodies, polynucleotides and linkers such as biotin.

Combinatorial library technology (Schultz, J S Biotechnol. Prog. 12, 729-743 (1996)) provides an efficient way of testing a potentially vast number of different substances for ability to modulate activity of a polypeptide.

The amount of test substance or compound which may be added to an assay of the invention will normally be determined by trial and error depending upon the type of compound used. Typically, from about 0.1 nM to 10 μM concentrations of a test compound (e.g. putative inhibitor) may be used. Greater concentrations may be used when a peptide is the test substance.

Compounds which may be used may be natural or synthetic chemical compounds used in drug screening programmes. Extracts of plants which contain several characterised or uncharacterised components may also be used.

Other inhibitor or candidate inhibitor compounds may be based on modelling the 3-dimensional structure of a polypeptide or peptide fragment and using rational drug design to provide potential inhibitor compounds with particular molecular shape, size and charge characteristics.

A substance which interacts with the polypeptide may be isolated and/or purified, manufactured and/or used to modulate its activity as discussed.

Following identification of a substance which modulates or inhibits mPGES-1 activity and/or inhibits fever, the substance may be investigated further. It may be formulated into a composition comprising at least one additional component such as a pharmaceutically acceptable excipient.

The inhibitor may be manufactured and/or used in preparation, i.e. manufacture or formulation, of a composition such as a medicament, pharmaceutical composition or drug. These may be administered to individuals.

The present invention provides a substance identified as an inhibitor of mPGES-1 activity and as an inhibitor of fever, in accordance with what is disclosed herein. The invention also provides a pharmaceutical composition, medicament, drug or other composition comprising such an inhibitor, a method comprising administration of such a composition to a patient, e.g. for treatment (which may include preventative treatment) of inflammation or other disease or condition as discussed, especially fever (pyretic response), use of such a substance in manufacture of a composition for administration, e.g. for treatment of fever, inflammation or other disease or condition as discussed, and a method of making a pharmaceutical composition comprising formulating the inhibitor into a composition comprising a pharmaceutical excipient, which may involve admixing such a substance with a pharmaceutically acceptable excipient, vehicle or carrier and optionally other ingredients.

The invention provides use of an inhibitor of mPGES-1 in the manufacture of a medicament for treating fever, and a pharmaceutical composition comprising an inhibitor of mPGES-1. The invention also provides a method of manufacturing a medicament for treating fever in an individual, comprising formulating an inhibitor of mPGES-1 into a composition comprising a pharmaceutical excipient.

A substance identified as an inhibitor of mPGES-1 and/or as an inhibitor of fever may be peptide or non-peptide in nature. Non-peptide “small molecules” are often preferred for many in vivo pharmaceutical uses. Accordingly, a mimetic or mimic of the inhibitor (particularly if a peptide) may be designed for pharmaceutical use. The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, e.g. peptides are not well suited as active agents for oral compositions as they tend to be quickly degraded by proteases in the alimentary canal. Mimetic design, synthesis and testing may be used to avoid randomly screening large number of molecules for a target property.

Mimetics may be developed prior to the first administration of the inhibitor to a test animal. Alternatively or additionally, mimetics may be developed or developed further following animal testing, and the developed mimetics tested in further animal trials.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in vivo or clinical testing.

Mimetics of substances identified as having ability to inhibit mPGES-1 and/or inhibit an inflammatory response in a screening method as disclosed herein are included within the scope of the present invention. A polypeptide, peptide or substance able to modulate activity of a polypeptide according to the present invention may be provided in a kit, e.g. sealed in a suitable container which protects its contents from the external environment. Such a kit may include instructions for use.

Further aspects and embodiments of the present invention will be apparent to those skilled in the art. The following experiments provide support for and exemplification by way of illustration of aspects and embodiments of the invention. All documents mentioned in this specification are incorporated by reference.

In the accompanying drawings:

FIG. 1 shows prostanoid synthesis during constitutive conditions and inflammation.

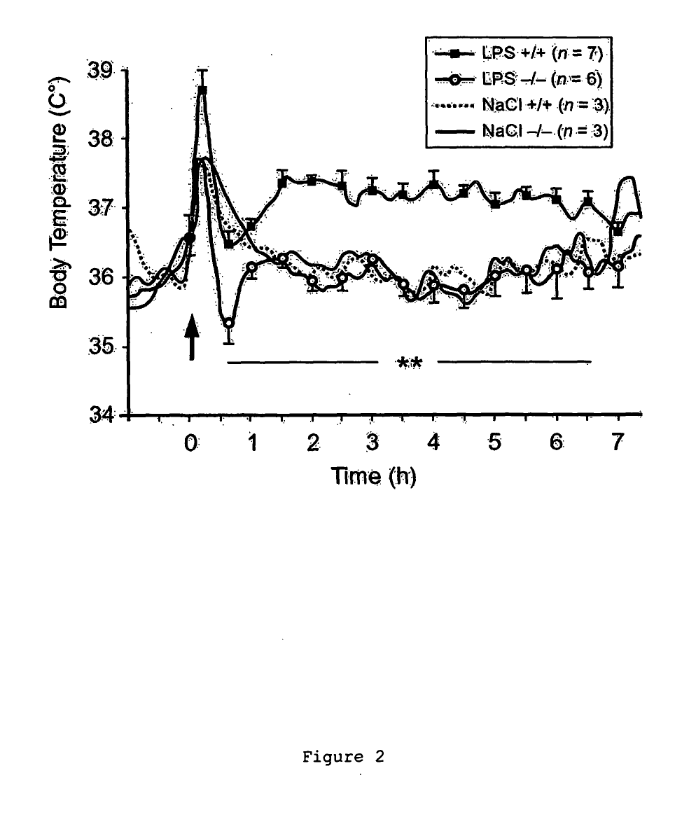

FIG. 2 shows febrile response following peripheral immune challenge. Intraperitoneal injection of LPS (arrow) resulted in robust temperature elevation in mPGES-1^{+/+} mice, but was absent in mPGES-1^{-/-} mice. Initial transient increase in body temperature was caused by the restraint during the injection procedure. Mice injected with LPS displayed a concomitant transient hypothermic reaction, not seen after saline injection. Error bars show SEM. **=P<0.01 (ANOVA and post hoc t-test; LPS treated +/+ mice vs. LPS-treated −/− mice).

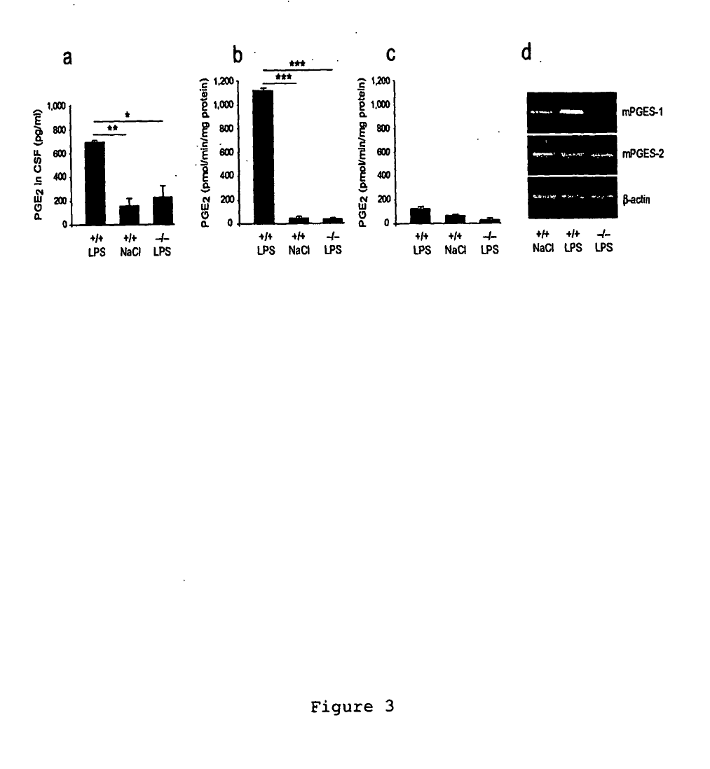

FIG. 3 shows PGE₂-levels and synthesis after immune challenge. (A) The concentration of PGE₂ in the cerebrospinal fluid (CSF) was significantly elevated in mPGES-1^{+/+} mice 3 h after intraperitoneal injection of LPS, whereas the PGE₂ levels in mPGES-1^{-/-} mice given LPS did not differ from those seen in mPGES-1^{+/+} mice injected with saline. (B) mPGES-1^{+/+} mice showed high enzymatic PGE₂-synthesising activity in the membrane fraction of brain homogenates incubated with PGH₂ 3 h after intraperitoneal injection of LPS, whereas PGE₂ formation in LPS-treated mPGES-1^{-/-} mice was low, as in mPGES-1^{+/+} mice given saline. (C) Low enzymatic PGE₂-synthesising activity was seen in the cytosolic fraction of brain homogenates incubated with PGH₂ 3 h after intraperitoneal injection of LPS, irrespective of genotype or treatment. (D) RT-PCR showed induced expression of mPGES-1 mRNA in mPGES-1^{+/+} mice but absence of transcript in mPGES-1^{-/-} mice 3 h after intraperitoneal injection of LPS. In contrast, mPGES-2 mRNA appeared down-regulated following LPS in wild type, but not in −/− mice. Error bars in (A) and (B) show SEM. *=P<0.05; **=P<0.01; ***=P<0.001 (Student's t-test).

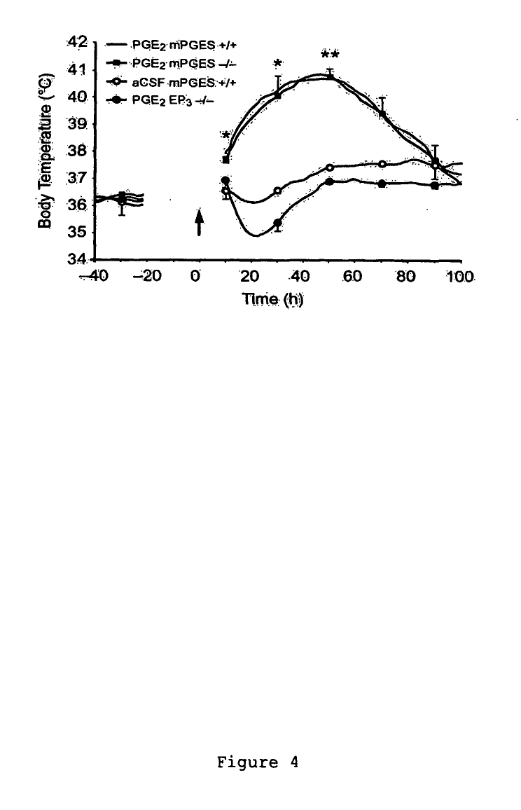

FIG. 4 shows PGE₂-induced febrile response. Intracerebroventricular injection of 4 nmol PGE₂ (arrow) resulted in rapid and pronounced temperature elevation in mPGES-1^{-/-} as well as in mPGES^{+/+} mice, but not in EP₃^{-/-} mice or in mPGES-1^{+/+} mice injected with artificial cerebrospinal fluid (aCSF). Error bars show SEM. *P<0.05, **P<0.01 (PGE₂-injected−/− mice vs. aCSF injected controls).

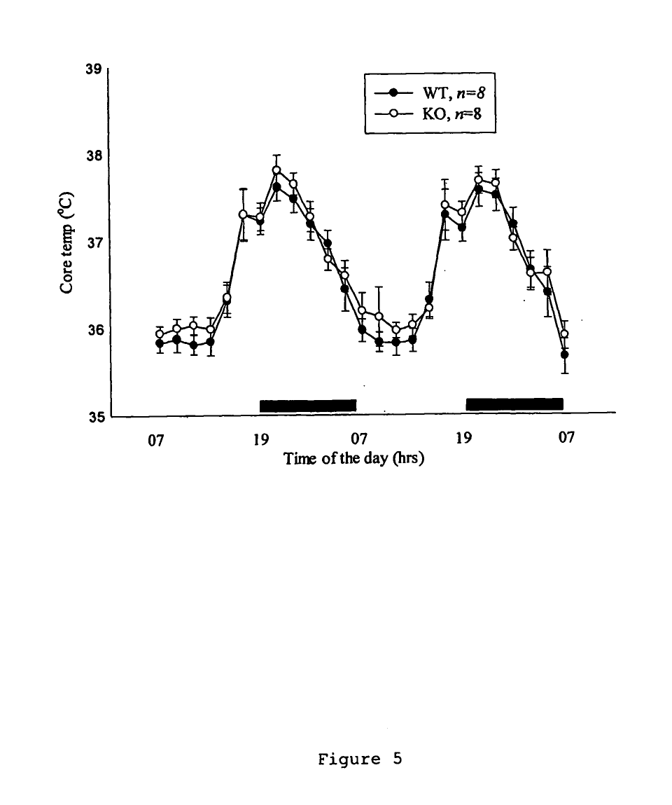

FIG. 5 shows diurnal temperature changes in wild type (WT) and Ptges^{-/-} (KO) mice (NB: Ptges is the gene that encodes mPGES-1. WT and KO mice displayed similar diurnal temperature variations. Each point represents mean±SEM. n=number of animals. Black bar along the abscissa indicates dark period. Mice were kept at an ambient temperature of 27±° C. There was no significant difference between the groups.

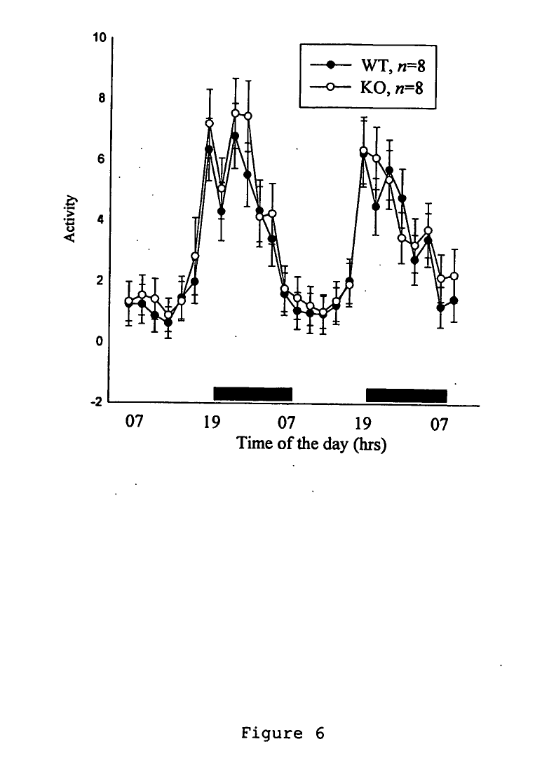

FIG. 6 shows diurnal variation in activity in wild type (WT) and Ptges-^{-/-} (KO) mice. Activity pattern in WT and KO mice varied with the light/dark periods in a similar way. Each point represents mean. n=number of animals. Black bar along the abscissa indicates dark period. There was no significant difference between the groups.

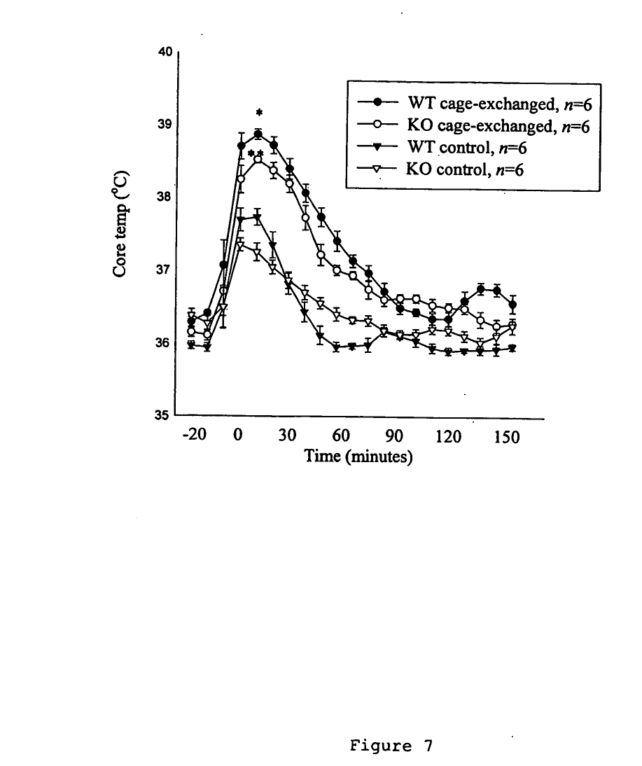

FIG. 7 shows the effect of cage exchange stress on the core temperature in wild type (WT) and Ptges^{-/-} (KO) mice. Cage exchange stress resulted in a hyperthermia in both WT and KO mice, which was more pronounced than that seen in control mice that were replaced in the own home cage. Each point represents mean±SEM. n=number of animals. 0 on the abscissa indicates time point when cages of two mice were exchanged, or for the controls, when the mice were replaced in their own home cage. Symbols represent level of significance when comparing peak temperature between cage exchanged and control mice within the WT (*=P<0.05) and KO (**=P<0.01) groups. There was no significant difference between WT and KO animals, neither for the cage exchanged or control groups.

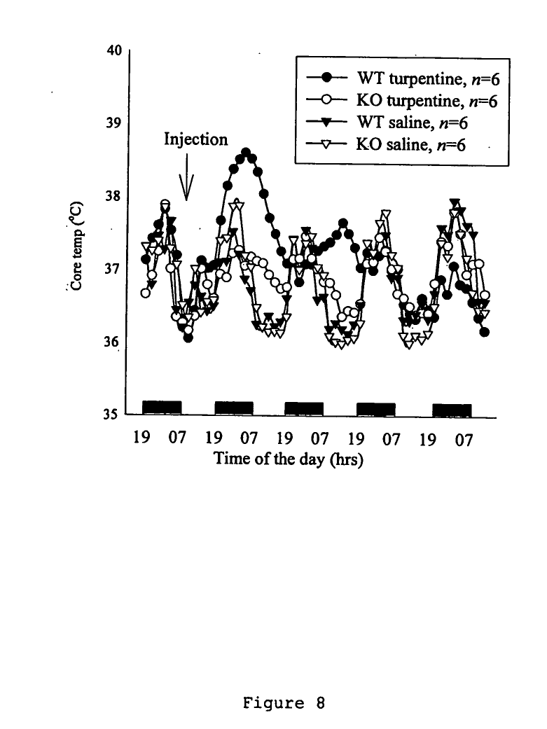

FIG. 8 shows the temperature responses to subcutaneous turpentine or saline injection in wild type (WT) and Ptges^{-/-} (KO) mice. Subcutaneous injection of turpentine in WT mice resulted in a febrile response during the first dark/light period after injection as well as during subsequent light period. KO mice injected with saline displayed a temperature curve that was similar to that of saline injected controls, but with an attenuated fall in core temperature during the beginning of second and third light periods after injection. Each point represents mean. n=number of animals. For the reason of perspicuity, error bars were omitted in this diagram. Average SEM was: WT turpentine, 0.28° C.; KO turpentine, 0.28° C.; WT saline, 0.59° C.; KO saline, 0.34° C. Black bar along the abscissa indicates dark period.

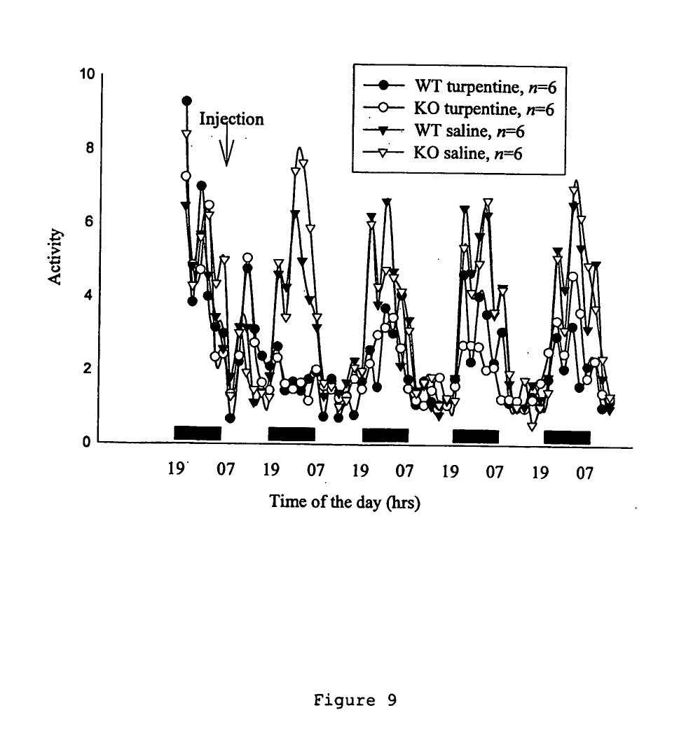

FIG. 9 shows the effects of turpentine injection on the activity patterns in wild type (WT) and Ptges^{-/-} (KO) mice. Subcutaneous injection of turpentine resulted in both WT and KO mice in an abolished activity increase during the first dark period after injection, and in an attenuated activity during the subsequent dark periods. Each point represents mean. n =number of animals. For the reason of perspicuity, error bars were omitted in this diagram. Average SEM was: WT turpentine, 0.80; KO turpentine, 0.80; WT saline, 0.97; KO saline, 1.02. Black bar along the abscissa indicates dark period.

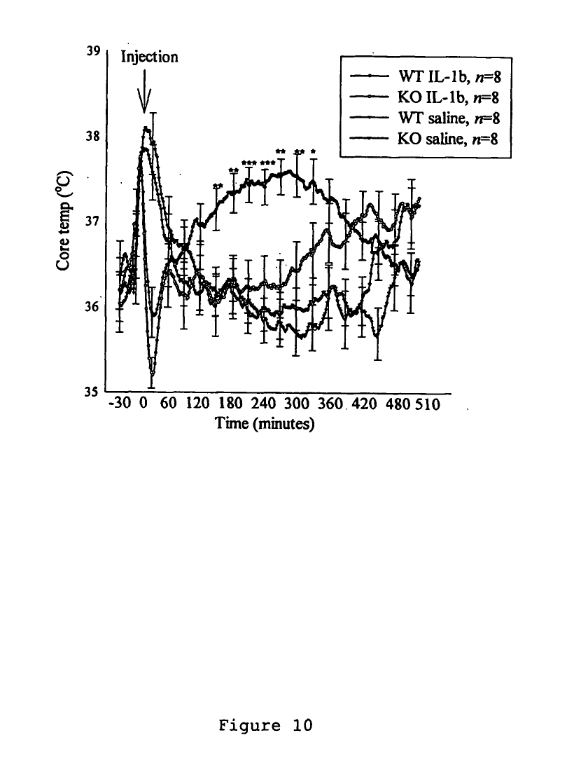

FIG. 10 shows the temperature responses to intraperitoneal injection of interleukin-1β (IL-1β) in wild type (WT) and Ptges^{-/-} (KO) mice. Following the injection-related hyperthermia and subsequent IL-1β induced hypotherma, WT mice given IL-1β displayed a rapid febrile response, in contrast to IL-1β injected KO mice and saline injected controls. Each point represents mean. For the reason of perspicuity, SEM is shown only for every 30 min. n=number of animals. * P<0.05; ** P<0.01; *** P<0.001 (IL-1 injected WT vs IL-1β injected KO mice).

### ABBREVIATIONS

12-HHT: 12(S)-Hydroxy-8,10-trans-5-cis-heptadecatrienoic acid
AA: arachidonic acid
COX-1: cyclooxygenase-1
COX-2: cyclooxygenase-2
FLAP: 5-lipoxygenase activating protein
LPS: Lipopolysaccharide
LT: Leukotriene
LTA₄, Leukotriene A₄: 5(S)-trans-5,6-oxido-7,9-trans-11,14-cis-eicosatetraenoic acid
LTC₄, Leukotriene C₄: 5(S)-hydroxy-6(R)-S-glutathionyl-7,9-trans-11,14-cis-eicosa-tetraenoic acid
mPGES-1: Microsomal protaglandin E synthase-1
Ptges: Gene encoding mPGES-1
MGST: microsomal glutathione S-tranferase
NSAID: Non-steroidal anti-inflammatory drug
PGD₂, Prostaglandin D₂: 9α, 15(S)-dihydroxy-11-ketoprosta-5-cis-13-trans-dienoic acid
PGE₂, prostaglandin E₂: 11α, 15(S)-dihydroxy-9-ketoprosta-5-cis-13-trans-dienoic acid
PGG₁, Prostaglandin G₁: 15(S)-hydroperoxy-9α, 11α-peroxidoprosta-13-enoic acid
PGG₂, Prostaglandin G₂: 15(S)-hydroperoxy-9α, 11α-peroxidoprosta-5-cis-13-trans-dienoic acid
PGG₃, Prostaglandin G₃: 15(S)-hydroperoxy-9α, 11α-peroxidoprosta-5,13,17-trienoic acid
PGH₁, Prostaglandin H₁: 15(S)-hydroxy-9α, 11α-peroxidoprosta-13-enoic acid
PGH₂, Prostaglandin H₂: 15(S)-hydroxy-9α, 11α-peroxidoprosta-5-cis-13-trans-dienoic acid
PGH₃, Prostaglandin H₃: 15(S)-hydroxy-9α, 11α-peroxidoprosta-5,13,17-trienoic acid
PGF_{2α}, Prostaglandin F_{2α}: 9α, 11α, 15(S)-trihydroxyprosta-5-cis-13-trans-dienoic acid
PGI₂, Prostacyclin: 6, 9α-epoxy-11α, 15(S)-dihydroxyprosta-5-cis-13-trans-dienoic acid
PGH synthase: Prostaglandin H synthase
RP-HPLC: Reverse-phase high performance liquid chromatography
TXA₂, Thromboxane A₂: 9α, 11α, epoxy-15(S)-hydroxythromba-5-cis-13-trans-dienoic acid

### EXPERIMENTS AND EMBODIMENTS

Febrile Response Following Immune Challenge by LPS-Induced Endotoxemia in Mice Deficient in mPGES-1

The febrile response was studied in inbred DBA/1l acJ mice bearing a targeted deletion of the mPGES-1 gene (Trebino, C. E. et al. Proc. Natl. Acad. Sci. USA 100, 9044-9049 (2003)). Heterozygous mice were bred to produce homozygous littermates. mPGES-1^{-/-} mice were born at predicted Mendelian frequency, grew normally, were fertile, and could not be distinguished in their general behavior from wild type (mPGES-1^{+/+}) mice (Trebino, C. E. et al. Proc. Natl. Acad. Sci. USA 100, 9044-9049 (2003)). The experiments were approved by the local Animal Care and Use Committee.

We first examined the febrile response of mPGES-1^{-/-} and mPGES-1^{+/+} mice to peripheral immune challenge. The animals were implanted with an intraperitoneal transmitter that permitted us to monitor their core temperature continuously by telemetry. A single dose of bacterial wall LPS (2 μg dissolved in 100 μl NaCl; Sigma 0111:B4), injected intraperitoneally one week after the implantation of the transmitter, elicited a robust temperature elevation in wild type animals (FIG. 2). It appeared at about 90 min after injection and lasted about 6 hours, which fits well with the time course of production of pyrogenic cytokines after intraperitoneal injection of LPS, although a direct endotoxin effect on brain endothelial cells cannot be ruled out (Laflamme, N., Lacroix, S. & Rivest, S. J. Neurosci. 19, 10923-10930 (1999)). In contrast, during the same time period mPGES-1^{-/-} mice displayed a core temperature that did not differ from that in mice that had been injected with saline (FIG. 2), showing that mPGES-1 is critical for endotoxin elicited fever. However, similar to wild type mice, the mPGES-1^{-/-} mice displayed a rapid but transient temperature increase immediately following the injection procedure, irrespective of whether LPS or saline was given (FIG. 2). Because this response, which is due to the restraint stress subjected to the animals during the injection procedure, was present also in mPGES-1^{-/-} mice, we conclude that it is independent of mPGES-1. It has also been shown to be independent of EP₃ receptors, the PGE₂ receptor subtype that seems to be critical for the thermogenic action of PGE₂ (Ushikubi, F. et al. Nature 395, 281-284 (1998)), providing an indication that it represents a PGE₂ independent mechanism.

Further, a transient hypothermic reaction that followed the restraint-induced hyperthermia was seen in both mPGES-1^{-/-} and mPGES-1^{+/+} mice that received LPS, but it was not seen in mice that received saline (FIG. 2). This hypothermic reaction, which is a common initial phenomenon upon immune challenge, was thus induced by LPS, possibly through peripheral vasodilatation elicited by macrophage-produced TNF-α (Wang, J., Ando, T. & Dunn, A. J. Neuroimmunomodulation 4, 230-236 (1997)), but was also independent of mPGES-1. Notably, the LPS induced fever in the mPGES-1^{+/+} mice was monophasic, thus differing from the biphasic curve recently reported in a study on wild-type C57/B6 mice (Oka, T. et al. J. Physiol. (Lond.) DOI:10.1113/jphysiol.2003.048140 (2003)), and the stress-induced initial hyperthermia was more pronounced than in that study. These differences are likely due to the use of different strains of mice, but could also reflect differences in ambient temperature as well in handling procedure before and during the injections.

We next analyzed PGE₂ levels in the brain of mPGES-1^{-/-} and mPGES-1^{+/+} mice in response, to the immune challenge. We found that the same intraperitoneal injection of LPS that elicited fever, also elicited a robust increase of PGE₂ in the cerebrospinal fluid in wild type mice (FIG. 3A), as has been shown previously in other species (Inoue, W. et al. Neurosci. Res. 44, 51-61 (2002)). In contrast, immune-challenged mPGES-1^{-/-} mice showed-concentrations of PGE₂ in the cerebrospinal fluid that did not differ from those displayed by wild type mice injected with saline (FIG. 3A).

To examine if the differences in cerebrospinal PGE₂ levels between immune-stimulated mPGES-1^{-/-} and mPGES-1^{+/+} mice were associated with differences in the PGE₂ synthesizing capacity, we focused on the PGES in situ enzymatic activity of these animals. Brain homogenates isolated from mPGES-1^{-/-} and mPGES-1^{+/+} mice injected intraperitoneally with LPS or saline were prepared, incubated with the PGES substrate, PGH₂, and analyzed for their PGE₂-synthetic capacity.

We found a strong enzymatic activity in the microsomal fraction of the brain homogenates in mPGES-1^{+/+} mice following intraperitoneal injection of LPS, whereas immune-challenged mPGES-1^{-/-} mice, similar to mPGES-1^{+/+} that were given NaCl intraperitoneally, displayed low activity (FIG. 3A). Low activity was also recorded in the cytosolic fraction of the brain homogenates, irrespective of genotype or treatment (FIG. 3C).

By using reverse transcriptase polymerase chain reaction we then examined the expression in the brain of the two microsomal prostaglandin E synthases, mPGES-1and mPGES-2. We confirmed that the mPGES-1^{-/-} mice were devoid of mPGES-1 mRNA, whereas wild type mice showed a strong induction of this enzyme after intraperitoneal LPS injection in comparison to wild type mice treated with saline (FIG. 3D). In contrast, mPGES-2 was not up-regulated by immune challenge (FIG. 3D). However, both mPGES-1^{-/-} and wild type mice showed a strong expression of inducible cox-2 following injection of LPS intraperitoneally.

Taken together, these data show that the deletion of the mPGES-1 gene was associated with an impaired PGE₂ production during immune challenge, but that it left the cox-2 expression intact. The data also indicate that other PGE-synthases are not involved in the LPS induced production of PGE₂.

We confirmed the absence of overt physiological signaling defects by ensuring that PGE₂-evoked (the missing product in mPGESl1^{-/-} mice that is generated following LPS challenge) pyresis was indeed intact in mPGES1^{-/-} mice. We performed intracerebroventricular injections of PGE₂ (4 nmol in 2 μl artificial cerebrospinal fluid) and then monitored using telemetry the body temperature in awake and unrestrained animals. We recorded a rapid and strong febrile response in the mPGES-1^{-/-} mice, similar to what we found in wild type littermates (FIG. 4). Thus, a pronounced temperature increase was seen almost immediately after the PGE₂ injection. It reached its peak after 30 minutes and lasted for about 1.5 hour. To determine that the elicited febrile response was due to specific activation of PGE₂ receptors and not represented an unspecific response, we injected the same amount of PGE₂ into EP₃ receptor^{-/-} mice as we had injected into mPGES-1^{-/-} mice. As expected, the mice with a targeted deletion of the EP₃ receptor gene did not display any febrile response to PGE₂ injected intracerebroventricularly (FIG. 4).

We conclude that the febrile response to endotoxin is critically dependent on induced PGE₂ production in the brain through the enzymatic action of mPGES-1. Because no fever developed in mice that lacked mPGES-1 following an immune challenge that mimics infectious and inflammatory conditions, pharmacological inhibition of mPGES-1 could similarly result in the inhibition of fever during such conditions.

In the light of the findings disclosed herein, we identify mPGES-1 as a potential novel drug target for the relief of fever and other PGE₂-mediated illness responses. In contrast to the presently available cox inhibitors, inhibition of mPGES-1 should leave the synthesis of other prostanoids intact as shown by our demonstration of retained induction of cyclooxygase-2 expression in the brain of mPGES-1^{-/-} mice. This may be an important advantage, since several of the severe side effects of cox inhibitors, and especially of the cox-2 specific drugs, may be due to their interference with the synthesis of other cox-dependent prostanoids, such as prostacyclin. Selective inhibition of mPGES-1 presents the possibility to treat fever and other centrally elicited acute phase reactions without many of the adverse side effects associated with presently available drugs.

Methods

Animals: Mice were kept one to a cage at constant ambient temperature of 25° C. and on a 12 h light/dark cycle (lights on at 7 a.m.). All experiments were performed under the early phase of the light cycle.

Telemetric temperature recordings: One week prior to each experiment, a temperature transmitter (Data Science International) was implanted into the peritoneal cavity of each mouse. Recordings were made via a receiver located beneath the cage and the data were fed into a computer. Recordings started at least one hour prior to surgery or injection, and were obtained every second minute throughout the entire session.

PGE₂ concentrations in the cerebrospinal fluid: Mice were killed by asphyxiation with CO₂ and cerebrospinal fluid was immediately removed by suboccipital puncture. The concentration of PGE₂ was determined by enzyme immunoassay, using a commercially available kit (Cayman) according to the manufacturer's instructions.

mPGES-1 activity: Brains were homogenized in 0.1 M KPi buffer containing 0.5 M sucrose, 1× complete protease inhibitor and 1 M GSH and centrifuged to remove insoluble material. The supernatant was centrifuged at 100,000×g for 2 h to separate the cytosolic and membrane fractions. Samples were diluted to a concentration of 2 mg protein/ml in a 0.1 M KPi buffer with 2.5 mM GSH. Prostaglandin E-synthase activity was assayed by adding 100 μl of each sample to 4 μl of 0.25 M PGH₂ for 1 min at 37° C. Following termination of the reaction, solid phase extraction was performed using C18 chromabond columns (Sigma). Formation of PGE₂ was determined by reverse phase high-performance liquid chromatography and UV detection at 195 nm. The enzymatic PGE₂ formation rate in the membrane fraction was calculated by subtracting the PGE₂ formation obtained in samples that had been pre-boiled to remove all enzymatic activity.

Reverse transcripts polymerase chain reaction: Total RNA was extracted using an RNeasy kit (Qiagen) and DNA was removed on column by incubation with DNase. RNA was reverse transcribed with the Superscript™ first strand synthesis system with oligo (dT) primers (Invitrogen), according to the manufacturer's instructions. The primer sequences were:

| | |
|---|---|
| mPGES-1: | |
| ctttctgctctgcagcacact | (SEQ ID NO: 3) |
| and | |
| gccatggagaaacaggagaac; | (SEQ ID NO: 4) |
| mPGES-2: | |
| ctatcaggtggtggaggtgaa | (SEQ ID NO: 5) |
| and | |
| cggacaatgtagtcgaaggaa; | (SEQ ID NO: 6) |
| β-actin: | |
| cttttcaaccagcagttccag | (SEQ ID NO: 7) |
| and | |
| cggacaccccttcacattatt. | (SEQ ID NO: 8) |

Intracerebroventricular injections of PGE₂: Mice were mounted in a stereotaxic frame under anesthesia with 1% isoflurane in a 30/70% mixture of O₂/N₂, and kept at 37° C. through a feedback controlled heating pad. A drill hole was made in the skull through which a 29 gauge (o.d.) needle connected by a silicon tube to a Hamilton syringe was inserted into the lateral ventricle (0.5 mm posterior to Bregma, 1 mm lateral to midline, and 2.5 mm vertical to the skull surface), and 4 nmol PGE₂ in 2 μl artificial cerebrospinal fluid was injected during 1 min. Control injection consisted of artificial cerebrospinal fluid. Two minutes after the end of the injection, the needle was removed, the skin sutured, and the gas anesthesia turned off. All animals were awake within 5 min after injection and were immediately returned to their home cage for resumed body temperature recordings.

Further Notes on PGE Synthase Activity Assays

Earlier studies have demonstrated that prostaglandins can be separated by RP-HPLC and detected by UV spectrophotometry (Terragno et al. Prostaglandins 21 (1), 101-12 (1981); Powell Anal. Biochem. 148(1), 59-69 (19.85)). The molar extinction coefficient of PGE2 is 16,500 at 192.5 nm (Terragno et al. Prostaglandins 21(1), 101-12 (1981)). The differences between the absorbance at 192.5 nm and 195 nm was marginal (Terragno et al. Prostaglandins 21(1), 101-12 (1981)). However, our results using the RP-HPLC conditions (described below) demonstrated a significantly more stable baseline, with less noise at the higher wavelength. The main products of PGH2 are PGF2α, PGE₂ and PGD₂. Using the described RP-HPLC conditions, the retention times were 19.0, 23.8 and 28.6 minutes for PGF2α, PGE₂ and PGD₂, respectively. In order to obtain an internal standard we have tested 11β-PGE2 and 16,16-dimethyl PGE₂. The latter compound was too hydrophobic and could not be used in the described isocratic system. In contrast, 11β-PGE₂ eluted with a retention time of 25.3 min with almost baseline separation from PGE₂. In order to investigate the UV-absorbance relationship between 11-β PGE₂ and PGE₂, equal amounts (quantified by GC-MS) were analyzed by RP-HPLC and analyzed by UV-absorbance at 195 nm. The two compounds showed identical UV-absorbance properties. In order to test the recovery and reproducibility of solid phase extraction, known amounts of 11-β PGE₂ and PGE₂ were diluted in sample buffer and acidified by adding the stop solution (containing no iron chloride) followed by the addition of acetonitrile (33% final cone) and subjected to analysis (10% (v/v) of total sample). Alternatively, after the addition of stop solution, the sample was extracted by solid phase extraction and the corresponding fraction (10% (v/v) of total sample) was then analysed. The amounts of 11-β PGE₂ and PGE₂ before and after extraction were compared and the recovery was estimated to be 85-90%.

In order to quantify PGE₂, a standard curve of PGE₂ was made. The curve was linear over the range from 0.9 pmol to 706 pmol (R²=0.9997, k=0.0012). For quantification we routinely use both the external standard as well as the internal standard technique, the latter method accounting also for losses during preparation.

Care must be taken when assaying PGE synthase with PGH₂. The substrate is very labile and decomposes non-enzymatically, with a half-life of about 5 min at 37° C., into a mixture of PGE₂ and PGD₂ with a E/D ratio of abut 3 (Hamberg et al. Proc. Natl. Acad. Sci. USA 71, 345-349 (1974); Nugteren and Christ-Hazelhof In Adv. in Prostaglandin and Thromboxane Res. 6, edited by B. Samuelsson, P. W. Ramwell, and R. Paoletti. Raven Press: NY, 129-137 (1980)). Also, the PGE synthase catalysis is very fast, which is why substrate depletion easily can occur within seconds thus preventing a quantitative analysis. After the reaction has been terminated, any remaining PGH₂ must also rapidly be separated from the products in order not to interfere with the results. To cope with these properties of the substrate, the assay may be performed as follows.

In order to minimize non-enzymatic production of PGE₂, the substrate (PGH₂) was always kept on CO₂-ice (−78° C.) until use and the enzyme reaction was performed at 0° C. in the presence of PGH₂ and reduced glutathione (GSH). A stop-solution was used, containing FeCl₂, which converted any remaining PGH₂ into HHT (Hamberg and Samuelsson Proc. Natl. Acad. Sci. USA 71(9), 3400-4) (1974). Also, the products are much more stable in organic solvents (Nugteren and Christ-Hazelhof In Adv. in Prostaglandin and Thromboxane Res. 6, edited by B. Samuelsson, P. W. Ramwell, and R. Paoletti. Raven Press: NY, 129-137 (1980)), so we immediately extracted the sample after termination by solid phase extraction and kept the eluate in acetonitrile.

Assay Method

Protein samples were diluted in potassium inorganic phosphate buffer (0.1M, pH 7.4) containing 2.5 mM reduced glutathione (GSH). 4 μl PGH₂, dissolved in acetone (0,284 mM) was added to eppendorf tubes and kept on CO₂-ice (−78° C.). Prior to the incubation, both the substrate and samples were transferred onto wet-ice (or 37° C.) for 2 min temperature equilibration. the reaction was started by the addition of the 100 μl sample to the tubes containing PGH₂. The reaction was terminated by the addition of 400 μl stop solution (25 mM FeCl₂, 50 mM citric acid and 2.7 μM 11-β PGE₂), lowering the pH to 3, giving a total concentration of 20 mM FeCl₂, 40 mM citric acid and 2.1 μM 11-β PGE₂. Solid phase extraction was performed immediately using C18-chromabond columns. The samples were eluted with 500 μl acetonitrile and thereafter 1 ml H₂O was added. In order to determine the formation of PGE₂ and 11-β PGE₂, an aliquot (150 μl) was analyzed by RP-HPLC, combined with UV detection at 195 nm. The reverse-phase HPLC column was Nova-Pak C18 (3.9×150 mm, 4 μm particle size) obtained from Waters and the mobile phase was water, acetonitrile and trifluoroacetic acid (72:28:0.007, by vol). The flow rate was 0.7 ml/min and the products were quantified by integration of the peak areas.

Febrile Response Following Immune Challenge by Aseptic, Ccytokine-Dependent Peripheral Inflammation and by Interleukin-1β in Mice Deficient in mPGES-1

The observations described above demonstrate that induced PGE₂ production by mPGES-1 is necessary for LPS-induced fever. The following observations show that this is a general mechanism for fever during immune challenge, i.e. it is elicited not only during endotoxemia, but also during cytokine-mediated immune responses.

LPS is known to elicit a cascade of cytokine synthesis, including the formation of interleukin-1β, interleukin-6 and tumor necrosis factor-α (TNF-α), and functional interleukin-1 type 1 receptors are expressed on the PGE₂-synthesizing endothelial cells (Konsman et al. J. Comp. Neurol. 472, 113-129 (2004)). Hence, while it is possible that the LPS-induced fever is cytokine-mediated, LPS could also exert its effect directly by binding to Toll-like receptor 4 (TLR4), which is expressed on cells of the leptomeninges, choroid plexus, and circumventricular organs, although not on the endothelial cells (Laflamme and Rivest, Faseb. J. 15, 155-163 (2001)), thus bypassing the cytokine pathway. In support of the latter idea, it has been shown that inhibitory-factor kappa Bα, an index of nuclear factor kappaB (NF-κB) activity, and Cox-2 transcripts are expressed in the endothelial cells of the brain vasculature after LPS challenge also in interleukin-1 deficient mice (Laflamme et al. J. Neurosci 19, 10923-10930 (1999)), and that TLR4-mutated mice are endotoxin resistant (Qureshi et al. J. Exp. Med. 189, 615-625 (1999)).

Therefore, in the present study, using mPGES-1 deficient mice, we examined the febrile response in an animal model that is dependent on intact cytokine signaling, namely the aseptic inflammation induced by subcutaneous injection of turpentine (Fantuzzi and Dinarello, J. Leukoc Biol. 59, 489-493 (1996)). We also studied the febrile response in the mPGES-1 deficient mice following intraperitoneal administration of interleukin-1β, and we examined the role of mPGES-1 in the circadian temperature variation and in stress-induced hyperthermia.

Materials and Methods

Animals. Mice with a deletion of the Ptges gene, which encodes mPGES-1, were generated by breeding heterozygous littermates of the DBA/lacJ strain, as previously reported (Trebino et al. Proc. Natl. Acad. Sci. USA 100, 9044-9049 (2003)). The animals were kept one per cage in a pathogen free facility at an ambient temperature of 27±1° C. and on a 12 h light/dark cycle (lights on at 7 a.m.), with food and water available ad libitum. All experimental procedures were performed during the early phase of light cycle.

Telemetric recordings. At least on week prior to the experiments, the mice were briefly anesthetized with isoflurane and implanted in the peritoneal cavity with a transmitter that records core temperature and motor activity (Data Science International, St. Paul, Minn. USA). A receiver, which transmits the signals on line to the connected computer, was placed beneath each cage. The animal's motor activity was qualitatively assessed from the change in position of the transmitter in relation to the receiver and the speed with which movement occurred. The recordings were started at least 1 hr prior to injection and data were obtained every 2 minutes throughout the entire observation period. The temperature recordings were sampled during 10 sec, whereas the activity recordings show the activity during the entire 2 min period.

Circadian changes in core temperature and motor activity. Core temperature and activity were monitored for two consecutive days. Thereafter, the mice were used for cage exchange stress experiment and subcutaneous injection of turpentine.

Cage-exchange induced stress response. Cage-exchange stress was evoked by exchanging the home cages of two mice. Control mice were just lifted up and placed back in the same cages.

Subcutaneous injection of turpentine. At least one day after the cage exchange stress experiment, mice were briefly anesthetized with isoflurane and given a subcutaneous injection of 150 μl of commercial grade turpentine (VWR, Stockholm, Sweden) in the left thigh. Control animals mice were injected with 150 μl saline.

Intraperitoneal-injection of IL-1β. The animals were briefly restrained and injected intraperitoneally with 600 ng of recombinant mouse IL-1β expressed in E. coli (Pierce Chemical Company, Sweden) diluted in 100 μl 0.9% NaCl. Mice in the control groups received an equal volume of saline. None of these mice had been subjected to any previous experiments.

Statistical analysis. The values are presented as means±S.E.M. Significant differences in the turpentine experiment were assessed by a four-way analysis of variance (ANOVA), with light/dark period, 24 h cycle, “treatment” and animal as factors, followed by Tukey's pairwise comparisons and single degree freedom test. Animals were nested within “treatments”, and constituted a random factor whereas all other factors were regarded as fixed factors. “Treatments” constituted of either of four combinations: wild type mice+saline, wild type mice+turpentine, knock out mice +saline or knock out mice+turpentine. Statistical differences in the other experiments were assessed by two-tailed t-test. A difference was considered to be significant if P<0.05.

Results

Diurnal Changes in Core Temperature and Motor Activity

The recordings of the diurnal core temperature changes of wild type and mPGES-1 knockout mice displayed no significant differences between the two groups (FIG. 5). Also the motor activity displayed by these mice was nearly identical between the two groups, and the diurnal changes were very similar to those displayed by the temperature recordings (FIG. 6).

Stress Induced Response

The cage exchange procedure induced a rapid and pronounced hyperthermia that did not differ between wild type and mPGES-1 knockout mice. In both groups the temperature increase was significantly larger that that seen in the control animals that were placed back into their own home cages (FIG. 7). These responses were not related to the motor activity pattern (data not shown). Thus, although the mice that were subjected to a cage exchange displayed more motor activity than the control mice, increased activity was seen throughout the observation period, whereas the temperature elevation continuously subsided.

Responses to Subcutaneous Injection of Turpentine

Following subcutaneous injection of turpentine, wild type mice displayed a biphasic fever response (FIG. 8). The first fever period started about 9 hrs after injection and persisted throughout the following night-day cycle. While there was no difference in temperature between the turpentine-injected and the saline-injected (control) animals during the second dark period after injection, the turpentine injected wild type animals showed an elevated temperature during the subsequent light period. During the third day after injection, the body temperature curve of the turpentine injected wild type animals basically followed that of the control mice.

Four-way ANOVA showed that the observed differences were statistically significant. Thus, during the first two 24 h cycles (starting 7 p.m. on the day of injection) there were significant temperature differences between the light and dark periods (F_{1,60}=119.25; P<0.001), and between the first and second cycle (F_{1,60}=23.22; P<0. 001). Further analysis showed that there was a statistically significant difference between “treatments” (wild type mice+saline; wild type mice+turpentine, knock out mice+saline, knock out mice+turpentine) (F_{3,20}=28.35; P<0.001). Tukey's pairwise comparisons showed that turpentine injected wild type mice differed from all other groups (family error rate 0.05), whereas no significant differences were seen between turpentine injected knockout mice, saline injected knockout mice and saline injected wild type mice. The fever response of the turpentine injected wild type animals was more pronounced during the first than the second 24 h cycle (F_{1,15}=27.72; P<0.001).

In contrast to the fever displayed by the wild type mice that were injected with turpentine, the mPGES-1 knock out mice did not show any clear febrile response, but displayed a core temperature curve that was similar to that of control animals. The only difference observed was that these mice displayed a slightly higher core temperature than the controls during the first and second light periods following the day of injection. Thus, the fall in core temperature associated with the beginning of the light period was delayed and less pronounced. Three-way ANOVA using only light period data showed that the temperature difference during the light periods between turpentine-injected knock out mice and saline-treatment knock out mice was statistically significant (F_{1,10}=8.40; P=0.016).

While there thus was a pronounced difference in the temperature response to turpentine injection between wild type and mPGES-1 knock out mice, the activity recordings of these mice were very similar (FIG. 9). In contrast to saline injected wild type and mutant mice, which showed an activity pattern that followed the day/night cycle (high activity during the dark period and low activity during the light period), both wild type and mutant mice that had been injected with turpentine displayed very low activity during the first dark period (at the same low level as during the light periods) and a somewhat higher but still clearly diminished activity during the subsequent dark periods. Hence, the difference in temperature response to turpentine-injection between wild type and mutant mice was unrelated to differences in activity, and the turpentine-induced activity depression was independent of mPGES-1.

Four-way ANOVA showed that there were significant activity differences between the light and dark periods (F_{1,60}=120.39; P<0.001) during the first two 24 h cycles (starting 7 p.m. on the day of injection). Further analysis showed that there was a significant difference between “treatments” (F_{3,20}=3.78; P=0.027), and single degree of freedom test confirmed that there was a difference between turpentine and saline-injected mice (F_{1,20}=11.19; P=0.003), whereas no difference was seen between knock out and wild type mice (F_{1,20}=0.09; P=0.770). These differences were more pronounced during the first than during the second dark period (two-way ANOVA:F_{1,20}=7.17; P=0.014).

Responses to Intraperitoneal Injection of IL-1β

All animals, irrespective of genotype or type of injection (IL-1β or saline), displayed an initial stress-induced hyperthermia due to the restraint associated with the injection procedure (FIG. 10). In IL-1β injected mice (wild type as well as mutant) this initial hyperthermia was immediately followed by a hypothermic response, which was not seen in saline injected mice. About 60-90 min after injection at which time point the animals had returned to a pre-injection body temperature, IL-1β injected mice, started to display a febrile response. This peaked at about 4 hrs after injection, and started to disappear after 6-7 hrs. In contrast, IL-1β injected mPGES-1 knockout mice did not show any febrile response during the first 4-5 hrs, but displayed a temperature curve that was similar to that displayed by saline injected wild type and mutant mice. However, starting at about 4-5 hrs after injection, the IL-1β injected knock out mice showed an increasing body temperature, whereas no significant change was seen in the saline injected controls before the last hours of the light period when these mice also displayed a temperature increase. While the latter is consistent with the circadian dependent temperature regulation (FIG. 5), the raise of the body temperature in the IL-1β injected knock out mice occurred earlier.

Discussion

The results of the present study demonstrate that the inducible PGE₂-synthesizing terminal isomerase mPGES-1 is critical for the development of fever in models using peripherally administered or released cytokines, such as intraperitoneal injection of IL-1β or subcutaneous injection of turpentine. The fever elicited by aseptic inflammation induced by turpentine is known to be cytokine dependent. Mice deficient in IL-1β or in IL-1 type 1 receptor do not develop fever following turpentine injection, but also IL-6 is necessary for the turpentine-induced fever (Zheng et al. Immunity 3, 9-19 (1995); Leon et al. Am. J. Physiol. 271, R1668-1675 (1996); Horai et al. J. Exp. Med. 187, 1463-1475 (1998); Kozak et al. Ann. N.Y. Acad. Sci. 856, 33-47 (1998)), thus implying that the mPGES-1 mediated febrile response to turpentine is cytokine-driven. Taken together with the demonstration described above that mPGES-1 is necessary for endotoxin-induced fever, the present findings indicate that mPGES-1 induced PGE₂ synthesis is a general and obligatory mechanism for the febrile response to infectious and inflammatory processes.

Only recently the origin of the inflammatory induced PGE₂ synthesis in the brain has become clear. Studies using in situ hybridization histochemistry have shown that mPGES-1 is induced in endothelial cells in the venules of the brain vasculature in response to inflammatory stimuli, and the same cells also show an induced Cox-2 expression, and bear receptors for IL-1 type 1 receptors (Ek et al. Nature 410, 430-431 (2001); Yamagata et al. J. Neurosci. 21, 2669-2677 (2001); Engblom et al. J. Comp. Neurol. 452, 205-214 (2002); Konsman et al. J. Comp. Neurol. 472, 113-129 (2004)).

The results described here show that following immune stimuli, the concentration of PGE₂ increases in the brain of wild type animals but not in mPGES-1 knockouts, and this increase is associated with an induced PGE₂ synthesizing capacity in the brain and with the capacity to develop immune-induced fever. The results further show that induced PGE₂ synthesizing capacity can solely be ascribed to induced mPGES-1 expression, because the expression of the other PGE₂ synthesizing enzymes that so far have been identified, cytosolic PGE synthase (CPGES) and mPGES-2, are either unaffected or down-regulated in the brain after inflammatory stimuli (also shown by Guay et al. J. Biol. Chem. 279, 24866-24872 (2004)). These data, taken together with the observation that mPGES-1, in contrast to Cox-2, has little constitutive expression in the brain and, with the possible exception of cells in the paraventricular hypothalamic nucleus (see Engblom et al. J. Comp. Neurol. 452, 205-214 (2002)), is induced only in the endothelial cells, strongly suggest that these cells are the likely source of the inflammation-induced PGE₂.

The effects of the PGE₂ that is synthesized in response to a peripheral inflammatory stimulus are exerted via its cognate receptors. Studies using genetically modified mice have demonstrated that the EP₃ receptor is critical for the febrile response. Animals with a deletion of the EP₃ receptor gene do not develop fever in response to subcutaneous turpentine, or to intraperitoneal lipopolysacharide or IL-1β injection (Ushikubi et al. Nature 395, 281-284 (1998); Oka et al. J. Physiol. 551, 945-954 (2003)), and, as described above (and subsequently in Engblom et al. Nat. Neurosci. 6, 1137-1138 (2003)) they are also unresponsive to intracerebrally delivered PGE₂ (see also Ushikubi et al. Nature 395, 281-284 (1998)). Experiments with restricted injection of PGE₂ into the brain parenchyma have shown that the fever-producing region is localized along the ventromedial aspect of the preoptic area of the hypothalamus, and local injection of a cyclooxygenase inhibitor into the same sites can alleviate the fever produced by peripherally administered endotoxin (Scammell et al. Am. J. Physiol. 274, R783-789 (1998)). The preoptic region is richly vascularized, shows a dense IL-1 type 1 receptor expression, and displays a strong NFκB and Cox-2 response after immune stimulation, indicating a high rate of prostaglandin synthesis (Konsman et al. J. Comp. Neurol. 472, 113-129 (2004)). The preoptic region is also rich in EP₃ receptors, many of which are expressed on inhibitory GABAergic neurons that project to the brain stem raphe pallidus nucleus, where they are supposed to exert a tonic inhibitory effect. Upon PGE₂ binding to the EP₃ receptor on the preoptic neurons the inhibition is thought to be released, resulting in fever development via activation by the raphe neurons of sympathetic effectors (Nakamura et al. J. Neurosci. 22, 4600-4610 (2002); Yoshida et al. Eur. J. Neurosci. 18, 1848-1860 (2003)).

In the present experiments, subcutaneous injection of turpentine in wild type mice resulted in a biphasic febrile response that lasted about 48 hours. Thus, a high core temperature was seen during the first dark/light cycle following injection and then during the subsequent light period, whereas it did not differ from that recorded in control animals during the intervening second dark period. Some previous studies using turpentine have shown less pronounced temperature changes, more suggestive of a turpentine-induced suppression of the normal circadian variation in core temperature than of an actual febrile response (Oka et al. J. Physiol. 551, 945-954 (2003)). These differences may be due to differences in the ambient temperature at which the experiments were performed. The present study was carried out at an ambient temperature of about 27° C., which is close to the thermoneutral zone of mice (Krol and Speakman, J. Exp. Biol. 206, 4255-4266 (2003)).

In contrast to the wild type mice, mPGES-1 mutant mice that were injected with turpentine displayed a temperature curve that was similar to that displayed by saline-injected wild type and mutant mice. However, a slight difference was noted. Whereas in the control animals the core temperature decreased rapidly at the end of the dark period, this circadian related fall in body temperature was less pronounced, especially following the first, but to some extent also following the second dark period after injection. The significance of this difference is unclear. While it may indicate the presence of some additional temperature regulating mechanism that is mPGES-1 independent (see Engblom et al. J. Mol. Med. 80, 5-15 (2002)), it could also be secondary to e.g. changes in the activity patterns of these animals. Thus, both the wild type and mutant mice that were injected with turpentine showed very little activity during the first dark period after injection, and attenuated activity during the subsequent dark periods, indicating that the activity depression was mPGES-1 independent. A similar dissociation between fever and activity has been found for IL-6 knockout mice. While IL-6 mutants did not develop fever after turpentine administration, the IL-6 gene deletion did not prevent the lethargy associated with the turpentine abscess (Kozak et al. Am. J. Physiol. 272, R621-630 (1997)). It is possible that the activity depression is mediated by a different prostaglandin, such as PGD₂ (Terao et al. J. Neurosci. 18, 6599-6607 (1998a); Terao et al. Neuroreport 9, 3791-3796 (1998b)).

The responses to intraperitoneal injection of IL-1β were similar to those which we previously demonstrated in the same strains of mice after intraperitoneal lipopolysaccharide injection (Engblom D. et al. Nat. Neurosci. 6, 1137-1138 (2003)). After the initial restraint-induced hyperthermia, IL-1β injected animals showed a rapid hypothermic response that was not present in saline injected controls. This hypothermia is probably elicited by an IL-1β induced release of TNF-α and consequent peripheral vasodilatation (Leon, Front Biosci. 9, 1877-1888 (2004)). The wild type mice injected with IL-1β showed a subsequent monophasic fever, whereas the mutant IL-1β injected mice displayed a temperature curve that with the exception of the later time points was similar to that seen in saline injected animals. Since the end of the observation period coincides with the beginning of the dark period, the elevation of the body temperature seen at the late time points in the saline injected controls is consistent with a circadian dependent temperature increase (see FIG. 5). However, because the late temperature increase seen in the IL-1 injected knock out mice began earlier that that seen in the controls, it seems to be an unrelated phenomenon. While it thus may represent some additional IL-1β elicited temperature regulating mechanism that is mPGES-1 independent, it should be recalled that bolus injection of IL-1β represents an artificial situation that results in cytokine concentrations and profiles that may not occur during naturally appearing infectious and inflammatory conditions.

In contrast to dependence of mPGES-1 for the inflammatory-induced febrile response that is demonstrated in the present study, mPGES-1 does not seem to be critical for the normal body temperature or for maintaining normal circadian temperature variations. Thus, wild type and mutant mice showed identical baseline temperature curves. The two groups also showed identical activity patterns, which even in minor details mimicked the temperature curves (FIGS. 5 and 6). A normal circadian temperature rhythm is seen also in EP₁, and EP₃ receptor mutant mice (Oka et al. J. Physiol. 551, 945-954 (2003)), as well as in EP₂ receptor mutant mice (Engblom and Blomqvist, unpublished). Unless the EP4 receptor will be shown to be involved, these data seem to indicate that the normal temperature regulation is PGE₂ independent. Whether it could be dependent on other prostaglandins is at present not clear. Cyclooxygenase inhibitors were found to attenuate the night-time rise in temperature in rats (Scales and Kluger, Am. J. Physiol. 253, R306-313 (1987)), but were reported to have little effect on the core temperature of monkeys (Barney and Elizondo, J. Appl. Physiol. 50, 1248-1254 (1981)). Examination of the circadian temperature variation in cyclooxygase mutant mice could help answer this question.

As shown by the hyperthermia elicited by the restraint during the IL-1β injection, this response was also mPGES-1 independent, and the same was found for the temperature response elicited by the cage exchange experiment. This type of psychological stress previously has been reported to be attenuated by cyclooxygenase inhibitors (Morimoto et al. J. Physiol. 443, 421-429 (1991)). However, it was of the same magnitude in mPGES-1 mutant mice as in wild type mice. It is also independent of the EP₁ and EP₃ receptors (Oka et al. J. Physiol. 551, 945-954 (2003)), as well as of the EP₂ receptor (Engblom and Blomqvist, unpublished). Taken together, these data show that the mechanism of psychological stress-induced hyperthermia is distinct from that of immune challenge-induced fever.

### SUMMARY OF EXPERIMENTAL EXAMPLES

In summary, the findings disclosed herein show that mPGES-1 is critical for the development of fever during endotoxin challenge (and subsequently in Engblom et al. Nat. Neurosci. 6, 1137-1138 (2003)), turpentine-induced abscess, and intraperitoneal injection of IL-1β, but that it is unrelated to the diurnal temperature regulation and stress-induced hyperthermia. Thus, the present findings demonstrate that mPGES-1 plays a major role in both central and peripheral inflammatory responses, and thus represents a potential therapeutic target for novel anti-inflammatory drugs to treat PGE₂-dependent inflammatory responses, especially fever.

1. A method of identifying an inhibitor of fever, comprising
administering a candidate inhibitor to a test animal, wherein the candidate inhibitor is a substance that inhibits microsomal PGE synthase-1 activity; and
determining the level of febrile response to a stimulus in the test animal compared to the level of febrile response to the stimulus in a control animal,
whereby a lower febrile response in the test animal than in the control animal indicates that the candidate inhibitor inhibits fever.

2. A method according to claim 1, including an earlier stage of identifying a candidate inhibitor of fever, comprising:
incubating a microsomal PGE synthase-1 polypeptide and a test substance in the presence of a cyclic endoperoxide substrate of the PGE synthase under conditions in which the PGE synthase normally catalyses conversion of the cyclic endoperoxide substrate into a product which is the 9-keto, 11α hydroxy form of the substrate;
determining production of the product;
comparing said production of the product with production of the product in the absence of the test substance;
detecting a reduced production compared with production in the absence of the test substance indicates that the substance inhibits microsomal prostaglandin E synthase-1; and
thereby identifying the test substance as a candidate inhibitor of fever.

3. A method according to claim 2 wherein the PGE synthase and the test substance are incubated in the presence of reduced glutathione and PGH₂ under conditions in which PGE₂ is normally produced, and the method comprises determining production of PGE₂.

4. A method according to claim 1, further comprising determining whether a candidate inhibitor or test substance inhibits another enzyme, including:
incubating the test substance or candidate inhibitor, in the presence of an enzyme other than mPGES-1 under conditions in which the enzyme normally catalyses a reaction producing a product; and
determining that production of the product is not reduced compared to production of the product in the absence of the test compound or candidate inhibitor.

5. A method according to claim 1, further comprising an earlier step of
bringing into contact a microsomal PGE synthase-1 polypeptide and test substance; and
detecting interaction or binding between the polypeptide and the test substance.

6. A method according to claim 1, wherein the candidate inhibitor inhibits expression of a gene encoding microsomal PGE synthase-1 activity.

7. A method according to claim 6, including an earlier stage of identifying a candidate inhibitor of fever, comprising:
(a) contacting DNA containing the promoter of said gene with a test substance, wherein the promoter is operably linked to a gene;
(b) determining the level of gene expression from the promoter; and
(c) detecting a reduced level of gene expression in the presence of the test substance compared with the level of gene expression in the absence of the test substance in comparable conditions,
whereby the test substance is identified as a candidate inhibitor of fever.

8. A method according to claim 6, wherein the candidate inhibitor comprises nucleic acid complementary to a gene encoding mPGES-1 or a fragment thereof.

9. A method according to claim 6, wherein the candidate inhibitor comprises double stranded RNA corresponding to the sequence of a gene encoding mPGES-1 or a fragment thereof.

10. A method according to claim 1, wherein level of febrile response is determined by measuring body temperature of the animal.

11. A method according to claim 1, wherein the stimulus is a fever-producing stimulus selected from the group consisting of: lipopolysaccharide, a cytokine, a microorganism, an experimentally-induced peripheral aseptic lesion and an experimentally-induced infectious and/or inflammatory disease.

12. A method according to claim 11, wherein the disease is peritonitis or arthritis.

13. A method according to claim 1, comprising identifying the candidate inhibitor as an inhibitor of fever.

14. A method according to claim 13 further comprising formulating the inhibitor into a composition comprising at least one additional component.

15. A method according to claim 14 wherein the composition comprises a pharmaceutically acceptable excipient.

16. Use of an inhibitor of microsomal PGE synthase-1 in the manufacture of a medicament for treating fever, wherein the inhibitor comprises
(i) nucleic acid complementary to a gene encoding mPGES-1 or a fragment thereof;
(ii) double stranded RNA corresponding to the sequence of a gene encoding mPGES-1 or a fragment thereof; or
(iii) a ribozyme specific for the RNA sequence of mPGES-1.

17. Use according to claim 16, wherein the inhibitor is identified by a method as defined above.

18. A method of manufacturing a medicament for treating fever in an individual, comprising formulating an inhibitor of microsomal PGE synthase-1 into a composition comprising a pharmaceutical excipient, wherein the inhibitor comprises
(i) nucleic acid complementary to a gene encoding mPGES-1 or a fragment thereof;
(ii) double stranded RNA corresponding to the sequence of a gene encoding mPGES-1 or a fragment thereof; or
(iii) a ribozyme specific for the RNA sequence of mPGES-1.

19. A pharmaceutical composition comprising an inhibitor of microsomal PGE synthase-1, wherein the inhibitor comprises
(i) nucleic acid complementary to a gene encoding mPGES-1 or a fragment thereof;
(ii) double stranded RNA corresponding to the sequence of a gene encoding mPGES-1 or a fragment thereof; or
(iii) a ribozyme specific for the RNA sequence of mPGES-1.

20. A method of treating fever in an individual, comprising administering an inhibitor of microsomal PGE synthase-1 to the individual, wherein the inhibitor comprises
(i) nucleic acid complementary to a gene encoding mPGES-1 or a fragment thereof;
(ii) double stranded RNA corresponding to the sequence of a gene encoding mPGES-1 or a fragment thereof; or
(iii) a ribozyme specific for the RNA sequence of mPGES-1.

21. A method according to claim 20, wherein the inhibitor is identified by a method as defined above.
